SEQUENCE LISTING (389 sequences)

SEQ ID NO: 1 - protein, 132 aa, Artificial Sequence
  CDR3 of variable heavy chain
EVQVIQSGADVKKPGSSVKVSCKSSGGTFSKYAINWVRQAPGQGLEWMGGIIPILGIANYAQKFQGRVTITTDESTSTAYMELSSLRSEDTAVYYCARGWGREQLAPHPSQYYYYYYGMDVWGQGTTVTVSS

SEQ ID NO: 2 - protein, 23 aa, Artificial Sequence
  CDR3 of variable heavy chain
GWGREQFAPHPSQYYYYYYGMDV

SEQ ID NO: 3 - protein, 23 aa, Artificial Sequence
  CDR3 of variable heavy chain
GWGREQDAPHPSQYYYYYYGMDV

SEQ ID NO: 4 - protein, 23 aa, Artificial Sequence
  CDR3 of variable heavy chain
GWGREQAAPHPSQYYYYYYGMDV

SEQ ID NO: 5 - protein, 23 aa, Artificial Sequence
  CDR3 of variable heavy chain
GWGREQLAAHPSQYYYYYYGMDV

SEQ ID NO: 6 - protein, 23 aa, Artificial Sequence
  CDR3 of variable heavy chain
GWGREQLAPAPSQYYYYYYGMDV

SEQ ID NO: 7 - protein, 23 aa, Artificial Sequence
  CDR3 of variable heavy chain
GWGREQLAPNPSQYYYYYYGMDV

SEQ ID NO: 8 - protein, 23 aa, Artificial Sequence
  CDR3 of variable heavy chain
GWGREQYAPHPSQYYYYYYGMDV

SEQ ID NO: 9 - protein, 23 aa, Artificial Sequence
  CDR3 of variable heavy chain
GWGREQLAPHLSQYYYYYYGMDV

SEQ ID NO: 10 - protein, 23 aa, Artificial Sequence
  CDR3 of variable heavy chain
GWGREQFAPHLSQYYYYYYGMDV

SEQ ID NO: 11 - protein, 23 aa, Artificial Sequence
  CDR3 of variable heavy chain
GWGREQFAPHLWQYYYYYYGMDV

SEQ ID NO: 12 - protein, 23 aa, Artificial Sequence
  CDR3 of variable heavy chain
GWGREQFAPNLWQYYYYYYGMDV

SEQ ID NO: 13 - protein, 23 aa, Artificial Sequence
  CDR3 of variable heavy chain
GWGREQFSPNPWQYYYYYYGMDV

SEQ ID NO: 14 - protein, 23 aa, Artificial Sequence
  CDR3 of variable heavy chain
GWGREQFSPNLWQYYYYYYGMDV

SEQ ID NO: 15 - protein, 23 aa, Artificial Sequence
  CDR3 of variable heavy chain
GWGREQLAPHLWQYYYYYYGMDV

SEQ ID NO: 16 - protein, 23 aa, Artificial Sequence
  CDR3 of variable heavy chain
GWGREQLAPNLWQYYYYYYGMDV

SEQ ID NO: 17 - protein, 23 aa, Artificial Sequence
  CDR3 of variable heavy chain
GWGREQLAPAPWQYYYYYYGMDV

SEQ ID NO: 18 - protein, 23 aa, Artificial Sequence
  CDR3 of variable heavy chain
GWGREQFAAHPSQYYYYYYGMDV

SEQ ID NO: 19 - protein, 23 aa, Artificial Sequence
  CDR3 of variable heavy chain
GWGREQFAPAPSQYYYYYYGMDV

SEQ ID NO: 20 - protein, 23 aa, Artificial Sequence
  CDR3 of variable heavy chain
GWGREQLAAAPSQYYYYYYGMDV

SEQ ID NO: 21 - protein, 23 aa, Artificial Sequence
  CDR3 of variable heavy chain
GWGREQYAPAPSQYYYYYYGMDV

SEQ ID NO: 22 - protein, 23 aa, Artificial Sequence
  CDR3 of variable heavy chain
GWGREQYAAHPSQYYYYYYGMDV

SEQ ID NO: 23 - protein, 23 aa, Artificial Sequence
  CDR3 of variable heavy chain
GWGREQYAPHLSQYYYYYYGMDV

SEQ ID NO: 24 - protein, 23 aa, Artificial Sequence
  CDR3 of variable heavy chain
GWGREQXXXXXXQYYYYYYGMDV

SEQ ID NO: 25 - protein, 8 aa, Artificial Sequence
  CDR1 of variable heavy chain
GGTFSNYA

SEQ ID NO: 26 - protein, 7 aa, Artificial Sequence
  CDR2 of variable heavy chain
IPILGIA

SEQ ID NO: 27 - protein, 7 aa, Artificial Sequence
  CDR1 of variable light chain
QSVRNNY

SEQ ID NO: 28 - protein, 3 aa, Artificial Sequence
  CDR2 of variable light chain
NGS

SEQ ID NO: 29 - protein, 10 aa, Artificial Sequence
  CDR3 of variable light chain
QQYGNSRRVT

SEQ ID NO: 30 - protein, 604 aa, Hendra virus
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 31 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 32 - protein, 127 aa, Homo sapiens
QVQLVQSGAEVKKPGASVKVSCKASGGTFSSYAISWVRQAPGQGLEWMGGIIPIFGTANYAQKFQGRVTITADESTSTAYMELSSLRSGDTAVYYCARDPGGYSYGPYYYYYGMDVWGQGTTVTVSS

SEQ ID NO: 33 - protein, 25 aa, Homo sapiens [sequence omitted]

SEQ ID NO: 34 - protein, 8 aa, Homo sapiens
GGTFSSYA

SEQ ID NO: 35 - protein, 18 aa, Homo sapiens
ISWVRQAPGQGLEWMGGI

SEQ ID NO: 36 - protein, 7 aa, Homo sapiens
IPIFGTA

SEQ ID NO: 37 - protein, 38 aa, Homo sapiens
NYAQKFQGRVTITADESTSTAYMELSSLRSGDTAVYYC

SEQ ID NO: 38 - protein, 20 aa, Homo sapiens
ARDPGGYSYGPYYYYYGMDV

SEQ ID NO: 39 - protein, 11 aa, Homo sapiens
WGQGTTVTVSS

SEQ ID NO: 40 - protein, 111 aa, Homo sapiens
DIQMTQSPSSVSASVGDRVTITCRASQGIGPWLAWYQQKPGKAPKFLIYRASTLQSGVPSRFSGSGSGTDFTLTISSLQPEDFATYYCQQAHSFPFTFGPGTKVDIKRTVA

SEQ ID NO: 41 - protein, 26 aa, Homo sapiens [sequence omitted]

SEQ ID NO: 42 - protein, 6 aa, Homo sapiens
QGIGPW

SEQ ID NO: 43 - protein, 17 aa, Homo sapiens
LAWYQQKPGKAPKFLIY

SEQ ID NO: 44 - protein, 3 aa, Homo sapiens
RAS

SEQ ID NO: 45 - protein, 36 aa, Homo sapiens
TLQSGVPSRFSGSGSGTDFTLTISSLQPEDFATYYC

SEQ ID NO: 46 - protein, 9 aa, Homo sapiens
QQAHSFPFT

SEQ ID NO: 47 - protein, 14 aa, Homo sapiens
FGPGTKVDIKRTVA

SEQ ID NO: 48 - protein, 132 aa, Homo sapiens
EVQLVQSGAEVKKPGSSVKVSCKSSGGTFSNYAINWVRQAPGQGLEWMGGIIPILGIANYAQKFQGRVTITTDESTSTAYMELSSLRSEDTAVYYCARGWGREQLAPHPSQYYYYYYGMDVWGQGTTVTVSS

SEQ ID NO: 49 - protein, 25 aa, Homo sapiens
EVQLVQSGAEVKKPGSSVKVSCKSS

SEQ ID NO: 50 - protein, 8 aa, Homo sapiens
GGTFSNYA

SEQ ID NO: 51 - protein, 18 aa, Homo sapiens
INWVRQAPGQGLEWMGGI

SEQ ID NO: 52 - protein, 7 aa, Homo sapiens
IPILGIA

SEQ ID NO: 53 - protein, 38 aa, Homo sapiens
NYAQKFQGRVTITTDESTSTAYMELSSLRSEDTAVYYC

SEQ ID NO: 54 - protein, 25 aa, Homo sapiens
ARGWGREQLAPHPSQYYYYYYGMDV

SEQ ID NO: 55 - protein, 11 aa, Homo sapiens
WGQGTTVTVSS

SEQ ID NO: 56 - protein, 111 aa, Homo sapiens
EIVLTQSPGTLSLSPGERATLSCRASQSITNGRLAWYQQKPGQAPRLLIYGVSSRASGIPERFSGSGSGTDFTLTISRLEPEDFAVYYCQQYGSSVLFGPGTKVDIKRTVA

SEQ ID NO: 57 - protein, 26 aa, Homo sapiens [sequence omitted]

SEQ ID NO: 58 - protein, 7 aa, Homo sapiens
QSITNGR

SEQ ID NO: 59 - protein, 17 aa, Homo sapiens
LAWYQQKPGQAPRLLIY

SEQ ID NO: 60 - protein, 3 aa, Homo sapiens
GVS

SEQ ID NO: 61 - protein, 36 aa, Homo sapiens
SRASGIPERFSGSGSGTDFTLTISRLEPEDFAVYYC

SEQ ID NO: 62 - protein, 8 aa, Homo sapiens
QQYGSSVL

SEQ ID NO: 63 - protein, 14 aa, Homo sapiens
FGPGTKVDIKRTVA

SEQ ID NO: 64 - protein, 118 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDSRYHDAFDIWGQGTMVTVSS

SEQ ID NO: 65 - protein, 25 aa, Homo sapiens
EVQLVESGGGLIQPGGSLRLSCAAS

SEQ ID NO: 66 - protein, 8 aa, Homo sapiens
GFTVSSNY

SEQ ID NO: 67 - protein, 18 aa, Homo sapiens
MSWVRQAPGKGLEWVSVI

SEQ ID NO: 68 - protein, 6 aa, Homo sapiens
YSGGST

SEQ ID NO: 69 - protein, 38 aa, Homo sapiens
YYADSVKGRFTISRDNSKNTLYLQMNSLRAEDTAVYYC

SEQ ID NO: 70 - protein, 12 aa, Homo sapiens
ARDSRYHDAFDI

SEQ ID NO: 71 - protein, 11 aa, Homo sapiens
WGQGTMVTVSS

SEQ ID NO: 72 - protein, 116 aa, Homo sapiens
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLYTFGQGTKLEIKRTVA

SEQ ID NO: 73 - protein, 26 aa, Homo sapiens
DVVMTQSPLSLPVTPGEPASISCRSS

SEQ ID NO: 74 - protein, 11 aa, Homo sapiens
QSLLHSNGYNY

SEQ ID NO: 75 - protein, 17 aa, Homo sapiens
LDWYLQKPGQSPQLLIY

SEQ ID NO: 76 - protein, 3 aa, Homo sapiens
LGS

SEQ ID NO: 77 - protein, 36 aa, Homo sapiens
NRASGVPDRFSGSGSGTDFTLKISRVEAEDVGVYYC

SEQ ID NO: 78 - protein, 9 aa, Homo sapiens
MQALQTLYT

SEQ ID NO: 79 - protein, 14 aa, Homo sapiens
FGQGTKLEIKRTVA

SEQ ID NO: 80 - protein, 119 aa, Homo sapiens
QVQLQQSGAEVKKPGSSVKVSCKASGGTFSSYAISWVRQAPGQGLEWMGGIIPIFGTANYAQKFQGRVTITADESTSTAYMELSSLRSEDTAVYYCARESSWLDAFDIWGQGTMVTVSS

SEQ ID NO: 81 - protein, 25 aa, Homo sapiens
QVQLQQSGAEVKKPGSSVKVSCKAS

SEQ ID NO: 82 - protein, 8 aa, Homo sapiens
GGTFSSYA

SEQ ID NO: 83 - protein, 19 aa, Homo sapiens
ISWVRQAPGQGLEWMGGII

SEQ ID NO: 84 - protein, 6 aa, Homo sapiens
PIFGTA

SEQ ID NO: 85 - protein, 38 aa, Homo sapiens
NYAQKFQGRVTITADESTSTAYMELSSLRSEDTAVYYC

SEQ ID NO: 86 - protein, 12 aa, Homo sapiens
ARESSWLDAFDI

SEQ ID NO: 87 - protein, 11 aa, Homo sapiens
WGQGTMVTVSS

SEQ ID NO: 88 - protein, 116 aa, Homo sapiens
DVVMTQSPLSLSVTAGEPASISCRSSQSLLHSNGHIYLDWYLQKPGQSPQLLIYMASNRASGVPDRFSGSGSGTDFTLRINRVETEDVGIYYCMQSLHTTRTFGQGTKVEIKRTVA

SEQ ID NO: 89 - protein, 26 aa, Homo sapiens
DVVMTQSPLSLSVTAGEPASISCRSS

SEQ ID NO: 90 - protein, 11 aa, Homo sapiens
QSLLHSNGHIY

SEQ ID NO: 91 - protein, 17 aa, Homo sapiens
LDWYLQKPGQSPQLLIY

SEQ ID NO: 92 - protein, 3 aa, Homo sapiens
MAS

SEQ ID NO: 93 - protein, 36 aa, Homo sapiens
NRASGVPDRFSGSGSGTDFTLRINRVETEDVGIYYC

SEQ ID NO: 94 - protein, 9 aa, Homo sapiens
MQSLHTTRT

SEQ ID NO: 95 - protein, 14 aa, Homo sapiens
FGQGTKVEIKRTVA

SEQ ID NO: 96 - protein, 122 aa, Homo sapiens
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARVGGITGTADAFDIWGQGTMVTVSS

SEQ ID NO: 97 - protein, 24 aa, Homo sapiens
QVQLVQSGGGVVQPGRSLRLSCAA

SEQ ID NO: 98 - protein, 9 aa, Homo sapiens
SGFTFSSYA

SEQ ID NO: 99 - protein, 18 aa, Homo sapiens
MHWVRQAPGKGLEWVAVI

SEQ ID NO: 100 - protein, 7 aa, Homo sapiens
SYDGSNK

SEQ ID NO: 101 - protein, 40 aa, Homo sapiens
YYADSVKGRFTISRDNSKNTLYLQMNSLRAEDTAVYYCAR

SEQ ID NO: 102 - protein, 13 aa, Homo sapiens
VGGITGTADAFDI

SEQ ID NO: 103 - protein, 11 aa, Homo sapiens
WGQGTMVTVSS

SEQ ID NO: 104 - protein, 116 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPFTFGPGTKVDIKRTVA

SEQ ID NO: 105 - protein, 26 aa, Homo sapiens
DVVMTQSPLSLPVTLGQPASISCRSS

SEQ ID NO: 106 - protein, 11 aa, Homo sapiens
QSLVYSDGNTY

SEQ ID NO: 107 - protein, 17 aa, Homo sapiens
LNWFQQRPGQSPRRLIY

SEQ ID NO: 108 - protein, 3 aa, Homo sapiens
KVS

SEQ ID NO: 109 - protein, 36 aa, Homo sapiens
NRDSGVPDRFSGSGSGTDFTLKISRVEAEDVGVYYC

SEQ ID NO: 110 - protein, 9 aa, Homo sapiens
MQGTHWPFT

SEQ ID NO: 111 - protein, 14 aa, Homo sapiens
FGPGTKVDIKRTVA

SEQ ID NO: 112 - protein, 131 aa, Homo sapiens
QVQLQQSGAEVKKPGSSVKVSCKASGGTFSSYAISWVRQAPGQGLEWMGGIIPIFGTANYAQKFQGRVTITADKSTSTAYMELSSLRSEDTAVYYCARDQLAGYYYDSSGYHYYYYGMDVWGQGTTVTVSS

SEQ ID NO: 113 - protein, 25 aa, Homo sapiens
QVQLQQSGAEVKKPGSSVKVSCKAS

SEQ ID NO: 114 - protein, 8 aa, Homo sapiens
GGTFSSYA

SEQ ID NO: 115 - protein, 18 aa, Homo sapiens
ISWVRQAPGQGLEWMGGI

SEQ ID NO: 116 - protein, 7 aa, Homo sapiens
IPIFGTA

SEQ ID NO: 117 - protein, 38 aa, Homo sapiens
NYAQKFQGRVTITADKSTSTAYMELSSLRSEDTAVYYC

SEQ ID NO: 118 - protein, 24 aa, Homo sapiens
ARDQLAGYYYDSSGYHYYYYGMDV

SEQ ID NO: 119 - protein, 11 aa, Homo sapiens
WGQGTTVTVSS

SEQ ID NO: 120 - protein, 111 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIKRTVA

SEQ ID NO: 121 - protein, 26 aa, Homo sapiens [sequence omitted]

SEQ ID NO: 122 - protein, 6 aa, Homo sapiens
QSISSY

SEQ ID NO: 123 - protein, 17 aa, Homo sapiens
LNWYQQKPGKAPKLLIY

SEQ ID NO: 124 - protein, 3 aa, Homo sapiens
AAS

SEQ ID NO: 125 - protein, 36 aa, Homo sapiens
SLQSGVPSRFSGSGSGTDFTLTISSLQPEDFATYYC

SEQ ID NO: 126 - protein, 9 aa, Homo sapiens
QQSYSTPIT

SEQ ID NO: 127 - protein, 14 aa, Homo sapiens
FGQGTRLEIKRTVA

SEQ ID NO: 128 - protein, 120 aa, Homo sapiens
EVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCARDHVHGPDAFDIWGQGTMVTVSS

SEQ ID NO: 129 - protein, 24 aa, Homo sapiens
EVQLVQSGAEVKKPGASVKVSCKA

SEQ ID NO: 130 - protein, 9 aa, Homo sapiens
SGYTFTGYY

SEQ ID NO: 131 - protein, 17 aa, Homo sapiens
MHWVRQAPGQGLEWMGI

SEQ ID NO: 132 - protein, 8 aa, Homo sapiens
INPSGGST

SEQ ID NO: 133 - protein, 38 aa, Homo sapiens
SYAQKFQGRVTMTRDTSTSTVYMELSSLRSEDTAVYYC

SEQ ID NO: 134 - protein, 13 aa, Homo sapiens
ARDHVHGPDAFDI

SEQ ID NO: 135 - protein, 11 aa, Homo sapiens
WGQGTMVTVSS

SEQ ID NO: 136 - protein, 114 aa, Homo sapiens
SYELTQPPSASGTPGQRVTISCSGSSSNIGSNTVNWYQQLPGTAPKLLIYRNNQRPSGVPDRFSGSKSGTSASLAISGLQSEDEADYYCAAWDDSLHVVFGGGTKLTVLGQPKA

SEQ ID NO: 137 - protein, 25 aa, Homo sapiens
SYELTQPPSASGTPGQRVTISCSGS

SEQ ID NO: 138 - protein, 8 aa, Homo sapiens
SSNIGSNT

SEQ ID NO: 139 - protein, 17 aa, Homo sapiens
VNWYQQLPGTAPKLLIY

SEQ ID NO: 140 - protein, 3 aa, Homo sapiens
RNN

SEQ ID NO: 141 - protein, 36 aa, Homo sapiens
QRPSGVPDRFSGSKSGTSASLAISGLQSEDEADYYC

SEQ ID NO: 142 - protein, 10 aa, Homo sapiens
AAWDDSLHVV

SEQ ID NO: 143 - protein, 15 aa, Homo sapiens
FGGGTKLTVLGQPKA

SEQ ID NO: 144 - protein, 116 aa, Homo sapiens
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGAFDIWGQGTMVTVSS

SEQ ID NO: 145 - protein, 25 aa, Homo sapiens
EVQLVESGGGLVKPGGSLRLSCAAS

SEQ ID NO: 146 - protein, 8 aa, Homo sapiens
GFTFSDYY

SEQ ID NO: 147 - protein, 17 aa, Homo sapiens
MSWIRQAPGKGLEWVSY

SEQ ID NO: 148 - protein, 8 aa, Homo sapiens
ISSSGSTI

SEQ ID NO: 149 - protein, 38 aa, Homo sapiens
YYADSVKGRFTISRDNAKNSLYLQMNSLRAEDTAVYYC

SEQ ID NO: 150 - protein, 9 aa, Homo sapiens
ARVGGAFDI

SEQ ID NO: 151 - protein, 11 aa, Homo sapiens
WGQGTMVTVSS

SEQ ID NO: 152 - protein, 112 aa, Homo sapiens
NFMLTQPHSVSGSPGKTVTISCTRSSGSIASNYVQWYRQSPGSAPTTVIYEGYQRPSGVPDRFSGSIDSSSNSASLTISGLETEDEADYYCQSYDATNHQVVFGGGTKLTVL

SEQ ID NO: 153 - protein, 26 aa, Homo sapiens
NFMLTQPHSVSGSPGKTVTISCTRSS

SEQ ID NO: 154 - protein, 7 aa, Homo sapiens
GSIASNY

SEQ ID NO: 155 - protein, 17 aa, Homo sapiens
VQWYRQSPGSAPTTVIY

SEQ ID NO: 156 - protein, 3 aa, Homo sapiens
EGY

SEQ ID NO: 157 - protein, 38 aa, Homo sapiens
QRPSGVPDRFSGSIDSSSNSASLTISGLETEDEADYYC

SEQ ID NO: 158 - protein, 11 aa, Homo sapiens
QSYDATNHQVV

SEQ ID NO: 159 - protein, 10 aa, Homo sapiens
FGGGTKLTVL

SEQ ID NO: 160 - protein, 118 aa, Homo sapiens
QMQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGWFRDWYFDLWGRGTLVTVSS

SEQ ID NO: 161 - protein, 25 aa, Homo sapiens
QMQLQQWGAGLLKPSETLSLTCAVY

SEQ ID NO: 162 - protein, 8 aa, Homo sapiens
GGSFSGYY

SEQ ID NO: 163 - protein, 17 aa, Homo sapiens
WSWIRQPPGKGLEWIGE

SEQ ID NO: 164 - protein, 7 aa, Homo sapiens
INHSGST

SEQ ID NO: 165 - protein, 38 aa, Homo sapiens
NYNPSLKSRVTISVDTSKNQFSLKLSSVTAADTAVYYC

SEQ ID NO: 166 - protein, 12 aa, Homo sapiens
ARGWFRDWYFDL

SEQ ID NO: 167 - protein, 11 aa, Homo sapiens
WGRGTLVTVSS

SEQ ID NO: 168 - protein, 108 aa, Homo sapiens
DIQMTQSPSSLSASVGDRVTITCRASQDIRNDLGWYQQRPGKAPKLLIYAASSLQSGVPSRFSGSGSGTDFTLTISSLQPEDSATYFCLQDYQYPWTFGQGTKVEIKR

SEQ ID NO: 169 - protein, 26 aa, Homo sapiens [sequence omitted]

SEQ ID NO: 170 - protein, 6 aa, Homo sapiens
QDIRND

SEQ ID NO: 171 - protein, 17 aa, Homo sapiens
LGWYQQRPGKAPKLLIY

SEQ ID NO: 172 - protein, 3 aa, Homo sapiens
AAS

SEQ ID NO: 173 - protein, 36 aa, Homo sapiens
SLQSGVPSRFSGSGSGTDFTLTISSLQPEDSATYFC

SEQ ID NO: 174 - protein, 9 aa, Homo sapiens
LQDYQYPWT

SEQ ID NO: 175 - protein, 11 aa, Homo sapiens
FGQGTKVEIKR

SEQ ID NO: 176 - protein, 121 aa, Homo sapiens
EVQLVQSGGGLVQPGRSLRLSCAASGFTFDDYAMHWVRQAPGKGLEWVSAISGSGGSTYYADSVKGRFTISRDNSKNTLYLQMNSLRAEDTAVYYCASEGLPETDDAFDIWGQGTMVTVSS

SEQ ID NO: 177 - protein, 25 aa, Homo sapiens
EVQLVQSGGGLVQPGRSLRLSCAAS

SEQ ID NO: 178 - protein, 8 aa, Homo sapiens
GFTFDDYA

SEQ ID NO: 179 - protein, 17 aa, Homo sapiens
MHWVRQAPGKGLEWVSA

SEQ ID NO: 180 - protein, 8 aa, Homo sapiens
ISGSGGST

SEQ ID NO: 181 - protein, 38 aa, Homo sapiens; identical to SEQ ID NO: 69

SEQ ID NO: 182 - protein, 14 aa, Homo sapiens
ASEGLPETDDAFDI

SEQ ID NO: 183 - protein, 11 aa, Homo sapiens
WGQGTMVTVSS

SEQ ID NO: 184 - protein, 113 aa, Homo sapiens
DVVMTQSPLSLPVTPGEPASISCRSSQSLLYSDGYNYLDWYLQKPGQSPQLLIYLGSRRASGVPDRFSGSGSGTDFTLKINTVEAEDVGVYYCMQGVEIPFTFGPGTKVEIKR

SEQ ID NO: 185 - protein, 26 aa, Homo sapiens
DVVMTQSPLSLPVTPGEPASISCRSS

SEQ ID NO: 186 - protein, 11 aa, Homo sapiens
QSLLYSDGYNY

SEQ ID NO: 187 - protein, 17 aa, Homo sapiens
LDWYLQKPGQSPQLLIY

SEQ ID NO: 188 - protein, 3 aa, Homo sapiens
LGS

SEQ ID NO: 189 - protein, 36 aa, Homo sapiens
RRASGVPDRFSGSGSGTDFTLKINTVEAEDVGVYYC

SEQ ID NO: 190 - protein, 9 aa, Homo sapiens
MQGVEIPFT

SEQ ID NO: 191 - protein, 11 aa, Homo sapiens
FGPGTKVEIKR

SEQ ID NO: 192 - protein, 114 aa, Homo sapiens
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATEGADYWGQGTLVTVSS

SEQ ID NO: 193 - protein, 25 aa, Homo sapiens
QVQLVQSGAEVKKPGATVKISCKVS

SEQ ID NO: 194 - protein, 8 aa, Homo sapiens
GYTFTDYY

SEQ ID NO: 195 - protein, 17 aa, Homo sapiens
MHWVQQAPGKGLEWMGL

SEQ ID NO: 196 - protein, 8 aa, Homo sapiens
VDPEDGET

SEQ ID NO: 197 - protein, 38 aa, Homo sapiens
IYAEKFQGRVTITADTSTDTAYMELSSLRSEDTAVYYC

SEQ ID NO: 198 - protein, 7 aa, Homo sapiens
ATEGADY

SEQ ID NO: 199 - protein, 11 aa, Homo sapiens
WGQGTLVTVSS

SEQ ID NO: 200 - protein, 114 aa, Homo sapiens
DVVMTQSPLSLPVALGQPASISCRSSQSLVHSDGNTYLNWFQQRPGQSPRRLLYKVSNRESGVPDRFSGSGSGSDFTLKISRVEAEDVGIYYCMQGTHWPPITFGQGTRLEIKR

SEQ ID NO: 201 - protein, 26 aa, Homo sapiens
DVVMTQSPLSLPVALGQPASISCRSS

SEQ ID NO: 202 - protein, 11 aa, Homo sapiens
QSLVHSDGNTY

SEQ ID NO: 203 - protein, 17 aa, Homo sapiens
LNWFQQRPGQSPRRLLY

SEQ ID NO: 204 - protein, 3 aa, Homo sapiens
KVS

SEQ ID NO: 205 - protein, 36 aa, Homo sapiens
NRESGVPDRFSGSGSGSDFTLKISRVEAEDVGIYYC

SEQ ID NO: 206 - protein, 10 aa, Homo sapiens
MQGTHWPPIT

SEQ ID NO: 207 - protein, 11 aa, Homo sapiens
FGQGTRLEIKR

SEQ ID NO: 208 - protein, 114 aa, Homo sapiens
QVQLVQSGAEVKKPGATVKISCKVSGYTFTDYYMHWVQQAPGKGLEWMGLVDPEDGETIYAQKFQGRVTITADTSTNTAYMELSSLRSEDTAVYYCATDGADYWDQGTLGTVST

SEQ ID NO: 209 - protein, 25 aa, Homo sapiens
QVQLVQSGAEVKKPGATVKISCKVS

SEQ ID NO: 210 - protein, 8 aa, Homo sapiens
GYTFTDYY

SEQ ID NO: 211 - protein, 18 aa, Homo sapiens
MHWVQQAPGKGLEWMGLV

SEQ ID NO: 212 - protein, 8 aa, Homo sapiens
DPEDGETI

SEQ ID NO: 213 - protein, 37 aa, Homo sapiens
YAQKFQGRVTITADTSTNTAYMELSSLRSEDTAVYYC

SEQ ID NO: 214 - protein, 7 aa, Homo sapiens
ATDGADY

SEQ ID NO: 215 - protein, 11 aa, Homo sapiens
WDQGTLGTVST

SEQ ID NO: 216 - protein, 111 aa, Homo sapiens
QSVLTQPPSVSGAPGQTVTISCTGSSSNIGGDSDVHWYQQLPGSAPKLLIYGNRNRPSGVPDRFSGSRSGTSASLAVIGVQADDEADYYCQCYDSSLNGYVFGPGTKVIVL

SEQ ID NO: 217 - protein, 26 aa, Homo sapiens
QSVLTQPPSVSGAPGQTVTISCTGSS

SEQ ID NO: 218 - protein, 8 aa, Homo sapiens
SNIGGDSD

SEQ ID NO: 219 - protein, 17 aa, Homo sapiens
VHWYQQLPGSAPKLLIY

SEQ ID NO: 220 - protein, 3 aa, Homo sapiens
GNR

SEQ ID NO: 221 - protein, 36 aa, Homo sapiens
NRPSGVPDRFSGSRSGTSASLAVIGVQADDEADYYC

SEQ ID NO: 222 - protein, 11 aa, Homo sapiens
QCYDSSLNGYV

SEQ ID NO: 223 - protein, 10 aa, Homo sapiens
FGPGTKVIVL

SEQ ID NO: 224 - protein, 119 aa, Homo sapiens
QVQLQQSGAEVKKPGSSVKVSCKASGGTFSSYAISWVRQAPGQGLEWMGWTNPNSGGTNYAQKLQGRVTMTTDTSTSTAYMELRSLRSDDTAVYYCANYKLQSDAFDIWGQGTMVTVSS

SEQ ID NO: 225 - protein, 25 aa, Homo sapiens
QVQLQQSGAEVKKPGSSVKVSCKAS

SEQ ID NO: 226 - protein, 6 aa, Homo sapiens
GGTFSS

SEQ ID NO: 227 - protein, 20 aa, Homo sapiens
YAISWVRQAPGQGLEWMGWT

SEQ ID NO: 228 - protein, 7 aa, Homo sapiens
NPNSGGT

SEQ ID NO: 229 - protein, 38 aa, Homo sapiens [sequence omitted]

SEQ ID NO: 230 - protein, 12 aa, Homo sapiens
ANYKLQSDAFDI

SEQ ID NO: 231 - protein, 11 aa, Homo sapiens
WGQGTMVTVSS

SEQ ID NO: 232 - protein, 108 aa, Homo sapiens
DIQMTQSPSSLSASVGDRVTITCRASQDIGNFLVWFQQKPGKAPKSLIYAASRLQSGVPSRFSGSGSGTDFTLTISSLQPEDFATYYCQHYKSYPLTFGGGTKVEIKR

SEQ ID NO: 233 - protein, 26 aa, Homo sapiens [sequence omitted]

SEQ ID NO: 234 - protein, 7 aa, Homo sapiens
QDIGNFL

SEQ ID NO: 235 - protein, 16 aa, Homo sapiens
VWFQQKPGKAPKSLIY

SEQ ID NO: 236 - protein, 3 aa, Homo sapiens
AAS

SEQ ID NO: 237 - protein, 36 aa, Homo sapiens
RLQSGVPSRFSGSGSGTDFTLTISSLQPEDFATYYC

SEQ ID NO: 238 - protein, 9 aa, Homo sapiens
QHYKSYPLT

SEQ ID NO: 239 - protein, 11 aa, Homo sapiens
FGGGTKVEIKR

SEQ ID NO: 240 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAGPVGATTGTFDYWGQGTLVTVSS

SEQ ID NO: 241 - protein, 25 aa, Homo sapiens [sequence omitted]

SEQ ID NO: 242 - protein, 10 aa, Homo sapiens
GGTFSSYAIS

SEQ ID NO: 243 - protein, 16 aa, Homo sapiens
WVRQAPGQGLEWMGGI

SEQ ID NO: 244 - protein, 7 aa, Homo sapiens
IPIFGTA

SEQ ID NO: 245 - protein, 38 aa, Homo sapiens; identical to SEQ ID NO: 85

SEQ ID NO: 246 - protein, 15 aa, Homo sapiens
ARAGPVGATTGTFDY

SEQ ID NO: 247 - protein, 11 aa, Homo sapiens
WGQGTLVTVSS

SEQ ID NO: 248 - protein, 108 aa, Homo sapiens
DIVMTQSPGTLSLSPGERATLSCRASQSVSSSYLAWYQQKPGQAPRLLIYGASSRATGIPDRFSGSGSGTDFTLTISRLEPEDFAVYYCQQYGSSFTFGPGTKVDIKR

SEQ ID NO: 249 - protein, 26 aa, Homo sapiens
DIVMTQSPGTLSLSPGERATLSCRAS

SEQ ID NO: 250 - protein, 6 aa, Homo sapiens
QSVSSS

SEQ ID NO: 251 - protein, 18 aa, Homo sapiens
YLAWYQQKPGQAPRLLIY

SEQ ID NO: 252 - protein, 3 aa, Homo sapiens
GAS

SEQ ID NO: 253 - protein, 36 aa, Homo sapiens [sequence omitted]

SEQ ID NO: 254 - protein, 8 aa, Homo sapiens
QQYGSSFT

SEQ ID NO: 255 - protein, 11 aa, Homo sapiens
FGPGTKVDIKR

SEQ ID NO: 256 - protein, 124 aa, Homo sapiens
QVQLQQSGAEVKKPGSSVKVSCKASGGTFRSYAISWVRQAPGQGLEWMGGIIPIFGTANYAQKFQGRVTITADESTSTAYMELSSLRSEDTAVYYCARGSQSYDHYYYYGMDVWGQGTTVTVSS

SEQ ID NO: 257 - protein, 25 aa, Homo sapiens
QVQLQQSGAEVKKPGSSVKVSCKAS

SEQ ID NO: 258 - protein, 8 aa, Homo sapiens
GGTFRSYA

SEQ ID NO: 259 - protein, 18 aa, Homo sapiens
ISWVRQAPGQGLEWMGGI

SEQ ID NO: 260 - protein, 7 aa, Homo sapiens
IPIFGTA

SEQ ID NO: 261 - protein, 38 aa, Homo sapiens; identical to SEQ ID NO: 85

SEQ ID NO: 262 - protein, 17 aa, Homo sapiens
ARGSQSYDHYYYYGMDV

SEQ ID NO: 263 - protein, 11 aa, Homo sapiens
WGQGTTVTVSS

SEQ ID NO: 264 - protein, 109 aa, Homo sapiens
QSALTQPASVSGSPGQSITISCTGTSSDVGGYNYVSWYQQHPGKAPKLMIFDVSNRPSGVSNRLSGSKSGNTASLTISGLQAEDEADYYCSSYTSNTVVFGGGTKLTVL

SEQ ID NO: 265 - protein, 26 aa, Homo sapiens
QSALTQPASVSGSPGQSITISCTGTS

SEQ ID NO: 266 - protein, 8 aa, Homo sapiens
SDVGGYNY

SEQ ID NO: 267 - protein, 17 aa, Homo sapiens
VSWYQQHPGKAPKLMIF

SEQ ID NO: 268 - protein, 3 aa, Homo sapiens
DVS

SEQ ID NO: 269 - protein, 36 aa, Homo sapiens
NRPSGVSNRLSGSKSGNTASLTISGLQAEDEADYYC

SEQ ID NO: 270 - protein, 9 aa, Homo sapiens
SSYTSNTVV

SEQ ID NO: 271 - protein, 10 aa, Homo sapiens
FGGGTKLTVL

SEQ ID NO: 272 - protein, 116 aa, Homo sapiens
EVQLVQSGAEVKKPGSSVKVSCKASGGAFSSYAISWVRQAPGQGLEWMGGIIPIFGTANYAQKFQGRVTITADESTSTAYMELSSLRSEDTAVYYCARDSAGLGAWGQGTLVAVSS

SEQ ID NO: 273 - protein, 25 aa, Homo sapiens
EVQLVQSGAEVKKPGSSVKVSCKAS

SEQ ID NO: 274 - protein, 8 aa, Homo sapiens
GGAFSSYA

SEQ ID NO: 275 - protein, 18 aa, Homo sapiens
ISWVRQAPGQGLEWMGGI

SEQ ID NO: 276 - protein, 7 aa, Homo sapiens
IPIFGTA

SEQ ID NO: 277 - protein, 38 aa, Homo sapiens; identical to SEQ ID NO: 85

SEQ ID NO: 278 - protein, 9 aa, Homo sapiens
ARDSAGLGA

SEQ ID NO: 279 - protein, 11 aa, Homo sapiens
WGQGTLVAVSS

SEQ ID NO: 280 - protein, 108 aa, Homo sapiens
DIQMTQSPSSLSASVGDRVTITCRASQGISSALAWYQQKPGKAPKLLIYDASSLESGVPSRFSGSGSGTDFTLTISSLQPEDFATYYCQQFNSYPLTFGQGTRLEIKR

SEQ ID NO: 281 - protein, 26 aa, Homo sapiens [sequence omitted]

SEQ ID NO: 282 - protein, 6 aa, Homo sapiens
QGISSA

SEQ ID NO: 283 - protein, 17 aa, Homo sapiens
LAWYQQKPGKAPKLLIY

SEQ ID NO: 284 - protein, 3 aa, Homo sapiens
DAS

SEQ ID NO: 285 - protein, 36 aa, Homo sapiens
SLESGVPSRFSGSGSGTDFTLTISSLQPEDFATYYC

SEQ ID NO: 286 - protein, 9 aa, Homo sapiens
QQFNSYPLT

SEQ ID NO: 287 - protein, 11 aa, Homo sapiens
FGQGTRLEIKR

SEQ ID NO: 288 - protein, 120 aa, Homo sapiens
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHRESGPEFFQHWGQGTLVTVSS

SEQ ID NO: 289 - protein, 25 aa, Homo sapiens
QITLKESGPTLVKPTQTLTLTCTFS

SEQ ID NO: 290 - protein, 10 aa, Homo sapiens
GFSLSTSGVG

SEQ ID NO: 291 - protein, 17 aa, Homo sapiens
VGWIRQPPGKALEWLAL

SEQ ID NO: 292 - protein, 8 aa, Homo sapiens
IYWDDDKR

SEQ ID NO: 293 - protein, 37 aa, Homo sapiens
YSPSLKSRLTITKDTSKNQVVLTMTNMDPVDTATYYC

SEQ ID NO: 294 - protein, 12 aa, Homo sapiens
AHRESGPEFFQH

SEQ ID NO: 295 - protein, 11 aa, Homo sapiens
WGQGTLVTVSS

SEQ ID NO: 296 - protein, 113 aa, Homo sapiens
DVVMTQSPLSLPVTLGQPASISCNSSQSLVYSNGITYLNWFHQRPGQSPRRLIYQVSNWDSEVPDRFSGSGSATDFTLKISRVEADDVGIYYCMQGTHWPPTFGQGTRLEIKR

SEQ ID NO: 297 - protein, 26 aa, Homo sapiens
DVVMTQSPLSLPVTLGQPASISCNSS

SEQ ID NO: 298 - protein, 11 aa, Homo sapiens
QSLVYSNGITY

SEQ ID NO: 299 - protein, 17 aa, Homo sapiens
LNWFHQRPGQSPRRLIY

SEQ ID NO: 300 - protein, 3 aa, Homo sapiens
QVS

SEQ ID NO: 301 - protein, 36 aa, Homo sapiens
NWDSEVPDRFSGSGSATDFTLKISRVEADDVGIYYC

SEQ ID NO: 302 - protein, 9 aa, Homo sapiens
MQGTHWPPT

SEQ ID NO: 303 - protein, 11 aa, Homo sapiens
FGQGTRLEIKR

SEQ ID NO: 304 - protein, 132 aa, Homo sapiens; identical to SEQ ID NO: 1

SEQ ID NO: 305 - protein, 25 aa, Homo sapiens
EVQLVQSGAEVKKPGSSVKVSCKSS

SEQ ID NO: 306 - protein, 8 aa, Homo sapiens
GGTFSNYA

SEQ ID NO: 307 - protein, 18 aa, Homo sapiens
INWVRQAPGQGLEWMGGI

SEQ ID NO: 308 - protein, 7 aa, Homo sapiens
IPILGIA

SEQ ID NO: 309 - protein, 38 aa, Homo sapiens; identical to SEQ ID NO: 53

SEQ ID NO: 310 - protein, 25 aa, Homo sapiens
ARGWGREQLAPHPSQYYYYYYGMDV

SEQ ID NO: 311 - protein, 11 aa, Homo sapiens
WGQGTTVTVSS

SEQ ID NO: 312 - protein, 110 aa, Homo sapiens
EIVMTQSPGTLSLSPGERATLSCRASQSVASRYLAWYQHKPGLAPRLLIYGASTRATGIPDRFSGSGSGTDFTLTISRLEPEDFAVYYCQQYGRTPSVTFGGGTKVEIKR

SEQ ID NO: 313 - protein, 26 aa, Homo sapiens
EIVMTQSPGTLSLSPGERATLSCRAS

SEQ ID NO: 314 - protein, 7 aa, Homo sapiens
QSVASRY

SEQ ID NO: 315 - protein, 17 aa, Homo sapiens
LAWYQHKPGLAPRLLIY

SEQ ID NO: 316 - protein, 3 aa, Homo sapiens
GAS

SEQ ID NO: 317 - protein, 36 aa, Homo sapiens
TRATGIPDRFSGSGSGTDFTLTISRLEPEDFAVYYC

SEQ ID NO: 318 - protein, 10 aa, Homo sapiens
QQYGRTPSVT

SEQ ID NO: 319 - protein, 11 aa, Homo sapiens
FGGGTKVEIKR

SEQ ID NO: 320 - protein, 108 aa, Homo sapiens
EIVMTQSPGTPSLSPGERATLSCRASQSIRSTYLAWYQQKPGQAPRLLIYGASSRATGIPDRFSGSGSGTDFTLTISRLEPEDFAVYYCQQYGRSPSFGQGTKVEIKR

SEQ ID NO: 321 - protein, 26 aa, Homo sapiens
EIVMTQSPGTPSLSPGERATLSCRAS

SEQ ID NO: 322 - protein, 7 aa, Homo sapiens
QSIRSTY

SEQ ID NO: 323 - protein, 17 aa, Homo sapiens
LAWYQQKPGQAPRLLIY

SEQ ID NO: 324 - protein, 3 aa, Homo sapiens
GAS

SEQ ID NO: 325 - protein, 36 aa, Homo sapiens [sequence omitted]

SEQ ID NO: 326 - protein, 8 aa, Homo sapiens
QQYGRSPS

SEQ ID NO: 327 - protein, 11 aa, Homo sapiens
FGQGTKVEIKR

SEQ ID NO: 328 - protein, 110 aa, Homo sapiens
EIVMTQSPGTLSLAPGERATLSCWASQSVRNNYLAWYQQKPGQAPRLVIYNGSTRATGIPDRFSGSGSGTDFTLTISRLDPEDFAVYYCQQYGNSRRVTFGGGTKVEIKR

SEQ ID NO: 329 - protein, 26 aa, Homo sapiens
EIVMTQSPGTLSLAPGERATLSCWAS

SEQ ID NO: 330 - protein, 7 aa, Homo sapiens
QSVRNNY

SEQ ID NO: 331 - protein, 17 aa, Homo sapiens
LAWYQQKPGQAPRLVIY

SEQ ID NO: 332 - protein, 3 aa, Homo sapiens
NGS

SEQ ID NO: 333 - protein, 36 aa, Homo sapiens
TRATGIPDRFSGSGSGTDFTLTISRLDPEDFAVYYC

SEQ ID NO: 334 - protein, 10 aa, Homo sapiens
QQYGNSRRVT

SEQ ID NO: 335 - protein, 11 aa, Homo sapiens
FGGGTKVEIKR

SEQ ID NO: 336 - protein, 108 aa, Homo sapiens
EIVLTQSPGTLSLSPGERATLSCRASQSVSSSYLAWYQQKPGQAPRLLIYGTSTRATGIPDRFSGSGSGTDFTLTISRLEPEDFAVYYCQRYGSSPAFGQGTKVEIKR

SEQ ID NO: 337 - protein, 26 aa, Homo sapiens [sequence omitted]

SEQ ID NO: 338 - protein, 7 aa, Homo sapiens
QSVSSSY

SEQ ID NO: 339 - protein, 17 aa, Homo sapiens
LAWYQQKPGQAPRLLIY

SEQ ID NO: 340 - protein, 3 aa, Homo sapiens
GTS

SEQ ID NO: 341 - protein, 36 aa, Homo sapiens; identical to SEQ ID NO: 317

SEQ ID NO: 342 - protein, 8 aa, Homo sapiens
QRYGSSPA

SEQ ID NO: 343 - protein, 11 aa, Homo sapiens
FGQGTKVEIKR

SEQ ID NO: 344 - protein, 107 aa, Homo sapiens
DIQMTQSPATLSASIGDRVTITCRASQSISKWLAWYQQKPGKAPKLLIYKASTLESGVPSRFSGSGSGTDFTLTISSLQPDDFATYYCQQYINYATFGQGTKVEIKR

SEQ ID NO: 345 - protein, 26 aa, Homo sapiens
DIQMTQSPATLSASIGDRVTITCRAS

SEQ ID NO: 346 - protein, 6 aa, Homo sapiens
QSISKW

SEQ ID NO: 347 - protein, 17 aa, Homo sapiens
LAWYQQKPGKAPKLLIY

SEQ ID NO: 348 - protein, 3 aa, Homo sapiens
KAS

SEQ ID NO: 349 - protein, 36 aa, Homo sapiens
TLESGVPSRFSGSGSGTDFTLTISSLQPDDFATYYC

SEQ ID NO: 350 - protein, 8 aa, Homo sapiens
QQYINYAT

SEQ ID NO: 351 - protein, 11 aa, Homo sapiens
FGQGTKVEIKR

SEQ ID NO: 352 - protein, 110 aa, Homo sapiens; identical to SEQ ID NO: 328

SEQ ID NO: 353 - protein, 26 aa, Homo sapiens
EIVMTQSPGTLSLAPGERATLSCWAS

SEQ ID NO: 354 - protein, 7 aa, Homo sapiens
QSVRNNY

SEQ ID NO: 355 - protein, 17 aa, Homo sapiens
LAWYQQKPGQAPRLVIY

SEQ ID NO: 356 - protein, 3 aa, Homo sapiens
NGS

SEQ ID NO: 357 - protein, 36 aa, Homo sapiens; identical to SEQ ID NO: 333

SEQ ID NO: 358 - protein, 10 aa, Homo sapiens
QQYGNSRRVT

SEQ ID NO: 359 - protein, 11 aa, Homo sapiens
FGGGTKVEIKR

SEQ ID NO: 360 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKR

SEQ ID NO: 361 - protein, 26 aa, Homo sapiens [sequence omitted]

SEQ ID NO: 362 - protein, 6 aa, Homo sapiens
QSVSSY

SEQ ID NO: 363 - protein, 17 aa, Homo sapiens
LAWYQQKPGQAPRLLIY

SEQ ID NO: 364 - protein, 3 aa, Homo sapiens
DAS

SEQ ID NO: 365 - protein, 36 aa, Homo sapiens [sequence omitted]

SEQ ID NO: 366 - protein, 8 aa, Homo sapiens
QQRSNWPT

SEQ ID NO: 367 - protein, 11 aa, Homo sapiens
FGQGTKVEIKR

SEQ ID NO: 368 - protein, 110 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTITFGQGTRLEIKR

SEQ ID NO: 369 - protein, 26 aa, Homo sapiens [sequence omitted]

SEQ ID NO: 370 - protein, 7 aa, Homo sapiens
QSVSSSY

SEQ ID NO: 371 - protein, 17 aa, Homo sapiens
LAWYQQKPGQAPRLLIY

SEQ ID NO: 372 - protein, 3 aa, Homo sapiens
GAS

SEQ ID NO: 373 - protein, 36 aa, Homo sapiens [sequence omitted]

SEQ ID NO: 374 - protein, 10 aa, Homo sapiens
QQYGSSPTIT

SEQ ID NO: 375 - protein, 11 aa, Homo sapiens
FGQGTRLEIKR

SEQ ID NO: 376 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVFGQGTKLEIKR

SEQ ID NO: 377 - protein, 26 aa, Homo sapiens [sequence omitted]

SEQ ID NO: 378 - protein, 7 aa, Homo sapiens
QSVSSSY

SEQ ID NO: 379 - protein, 17 aa, Homo sapiens
LAWYQQKPGQAPRLLIY

SEQ ID NO: 380 - protein, 3 aa, Homo sapiens
GAS

SEQ ID NO: 381 - protein, 36 aa, Homo sapiens [sequence omitted]

SEQ ID NO: 382 - protein, 8 aa, Homo sapiens
QQYGSSPV

SEQ ID NO: 383 - protein, 11 aa, Homo sapiens
FGQGTKLEIKR

SEQ ID NO: 384 - protein, 23 aa, Artificial Sequence
  CDR3 of variable heavy chain
GWGREQFAAHPWQYYYYYYGMDV

SEQ ID NO: 385 - protein, 23 aa, Artificial Sequence
  CDR3 of variable heavy chain
GWGREQYAPAPWQYYYYYYGMDV

SEQ ID NO: 386 - protein, 23 aa, Artificial Sequence
  CDR3 of variable heavy chain
GWGREQFAPAPWQYYYYYYGMDV

SEQ ID NO: 387 - protein, 8 aa, Artificial Sequence
  CDR1 of variable heavy chain
GGTFSKYA

SEQ ID NO: 388 - protein, 25 aa, Homo sapiens
EVQVIQSGADVKKPGSSVKVSCKSS

SEQ ID NO: 389 - protein, 38 aa, Homo sapiens
NYAQKFQGRVTITTDESTSTAYMELSSLRSEDTALYYC

(19) United States
(12) Patent Application Publication
(10) Pub. No.: US 2014/0065166 A1
(43) Pub. Date: Mar. 6, 2014

(54) Neutralizing Antiboides to Nipah and Hendra Virus

(71) Applicants: Christopher C. Broder, Silver Spring, MD (US); Deborah L. Fusco, Silver Spring, MD (US); Kai Hu, New York, NY (US); Dimitar B. Nikolov, New York, NY (US)

(72) Inventors: Christopher C. Broder, Silver Spring, MD (US); Deborah L. Fusco, Silver Spring, MD (US); Kai Hu, New York, NY (US); Dimitar B. Nikolov, New York, NY (US)

(73) Assignee: The Henry M. Jackson Foundation for the Advancemen t of Military Medicine, Inc., Bethesda, MD (US)

(21) Appl. No.: 14/114,465

(22) PCT Filed: Apr. 9, 2013
(86) PCT No.: PCT/US13/35806

§ 371 Date: Oct. 28, 2013

Related U.S. Application Data
(60) Provisional application No. 61/480,161, filed on Apr. 28, 2011.

(51) Int. Cl. C07K 16/10

(52) U.S. Cl. CPC C07K 16/1027
USPC 424/159.1; 530/389.4; 536/23.53; 435/320.1; 435/339; 435/69.6

(57) ABSTRACT

The invention described herein provides novel peptides. The novel peptides are useful alone or as portions of larger molecules, such as antibodies or antibody fragments, that can be used to treat or prevent infection of Nipah virus and/or Hendra virus.

### BACKGROUND

Solid-state foams refer to foams made by a particular method where the process of introducing bubbles is carried out in the solid-state—just above the glass transition temperature of the polymer. The foams produced by this method typically have cell sizes in the 10-50 μm range, and are known as microcellular foams. There are continuing efforts to drive down the cell size to the 10-100 nm range to harness some of the unique properties predicted at this size range.

There are two basic steps in the solid-state foaming of thermoplastic polymers. The first step includes saturation of the polymer with gas under high pressure. This step is normally carried out at room temperature. Given sufficient time for diffusion of gas into the polymer, the gas attains an equilibrium concentration that is consistent with the solubility of gas in the polymer and the gas pressure. In the second step, bubbles are nucleated in the gas-polymer system by creating a thermodynamic instability. This is achieved by either a sudden drop in pressure or sudden increase in temperature. Both strategies suddenly reduce the solubility of the gas, driving the gas out of the polymer matrix and into nucleated bubbles. One consequence of dissolving gas in the polymer is plasticization, reducing the polymer's glass transition temperature. After saturation, the temperature of the gas-saturated polymer only needs to be raised to the glass transition temperature of the gas-polymer system to nucleate bubbles. The phrase “solid-state foam” is used to describe such foams, as opposed to other conventional foams produced from a polymer melt, such as via extrusion. This solid-state foaming process uses a benign or inert gas as the blowing agent instead of hazardous chemicals, and thus is environmentally friendly.

### SUMMARY

Disclosed is a method for creating a cellular thermoplastic material. In some embodiments, a method includes heating a solid, noncellular, gas-unsaturated, thermoplastic material to a temperature greater than the material's glass transition temperature, and below the melting temperature, during which the thermoplastic material remains a solid. Then, allowing the material to cool. After the material has cooled, saturating the cooled thermoplastic material with a non-reacting gas to provide a gas-saturated material, during which the material remains a solid. Thereafter, heating the gas-saturated material below the melting temperature of the material so that the material remains a solid, and causes nucleation of bubbles, and creation of cells in the material.

In some embodiments, the material can be thermoplastic polyurethane.

In some embodiments, the material can be polycarbonate, polystyrene, or polymethyl methacrylate.

In some embodiments, the solid noncellular material is formed by melting prior to heating.

In some embodiments, the residual stresses as a result of melting and cooling are reduced by subsequent heating and slow cooling.

In some embodiments, the material can be a sheet or film.

Some embodiments of a method for creating a cellular thermoplastic material, include, forming a solid, noncellular thermoplastic material by melting and introducing an additive into the material, wherein the additive lowers a surface energy of the material; after the material has solidified, saturating the solid thermoplastic material with a non-reacting gas to provide a solid gas-saturated material; and heating the gas-saturated solid material below the melting temperature of the material so that the material remains a solid and causes nucleation of bubbles and creation of cells in the material.

Also disclosed are embodiments of a thermoplastic foam made by the methods above.

In some embodiments, the thermoplastic foam can have a relative density of about 54% to about 57%.

In some embodiments, the thermoplastic foam can have an average cell size less than 7 μm. In some embodiments, the thermoplastic foam can have an average cell size in the range of 5 μm to 10 μm.

In some embodiments, the thermoplastic foam can have a cell nucleation density greater than 3×10⁹ cells/cm³. In some embodiments, the thermoplastic foam can have a cell nucleation density that ranges from about 3×10⁹ cells/cm³ to about 6×10⁹ cells/cm³.

Some embodiments of a method for creating a foam from a solid thermoplastic material include applying a process to lower the surface energy of a solid, noncellular, gas-unsaturated, thermoplastic material, while the material remains a solid. After lowering the surface energy, saturating the solid thermoplastic material with a non-reacting gas during which the material remains a solid and provides a gas-saturated solid material. After saturating the solid thermoplastic material, inducing the nucleation of bubbles, and creation of cells in the gas-saturated solid material, while the material remains a solid.

In some embodiments, the method includes heating the gas-saturated material below the melting temperature of the material so that the material remains a solid, and causes the nucleation of bubbles, and creation of cells in the material.

In some embodiments, the material has been formed by a melting and cooling process that introduces residual stresses in the material, which are thereafter reduced.

In some embodiments, the process to lower the surface energy comprises heating the material above the glass transition temperature of the material, but lower than the melting temperature, and then cooling the material.

In some embodiments, the process to lower the surface energy is to introduce additives into the material, such as during the forming process.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a flow diagram of a method for producing solid state foams, with an anneal process prior to saturation;

FIG. 2 is a micrograph of a comparative example of a foam created from an unannealed TPU (thermoplastic polyurethane) material;

FIG. 3 is a micrograph of an example of a foam created from an annealed TPU material;

FIG. 4 is a graph comparing the relative density of foams created from unannealed versus annealed materials;

FIG. 5 is a graph comparing the average cell size of foams created from unannealed versus annealed materials;

FIG. 6 is a graph comparing the cell nucleation density of foams created from unannealed versus annealed materials;

FIG. 7 is a graph comparing the gas concentration of unannealed versus annealed materials;

FIG. 8 is a graph showing the local variation in average cell size of comparative unannealed samples;

FIG. 9 is a graph showing the local variation in average cell size of annealed samples;

FIG. 10 is a graph showing the local variation in cell nucleation density for comparative unannealed samples;

FIG. 11 is a graph showing the local variation in cell nucleation density for annealed samples

FIG. 12A is a micrograph of a comparative example of a foam created from an unannealed 42D TPU material;

FIG. 12B is a micrograph of an example of a foam created from an annealed 42D TPU material;

FIG. 13A is a micrograph of a comparative example of a foam created from an unannealed 72D TPU material;

FIG. 13B is a micrograph of an example of a foam created from an annealed 72D TPU material;

FIG. 14A is a micrograph of a comparative example of a foam created from an unannealed PC (polycarbonate) material;

FIG. 14B is a micrograph of an example of a foam created from an annealed PC material;

FIG. 15A is a micrograph of a comparative example of a foam created from an unannealed PS (polystyrene) material; and

FIG. 15B is a micrograph of an example of a foam created from an annealed PS material.

### DETAILED DESCRIPTION

Disclosed is a process for increasing the cell density of materials via a solid-state foaming method as compared to previous solid-state foaming methods. In the disclosed process, an anneal (or heating) step is performed prior to saturation of materials with a saturating gas, and after the materials have been formed, such as via an extrusion process, which may also utilize heat to melt and shape the materials into the form that is annealed and saturated with gas.

Cell density is defined to mean the number of cells for a given volume. In one embodiment, it is believed that cell sizes in the 10-100 nm range can be created by the disclosed process. However, in other embodiments, cell sizes in the range of 100-500 nm, 500-1000 nm, and greater than 1000 nm can be created.

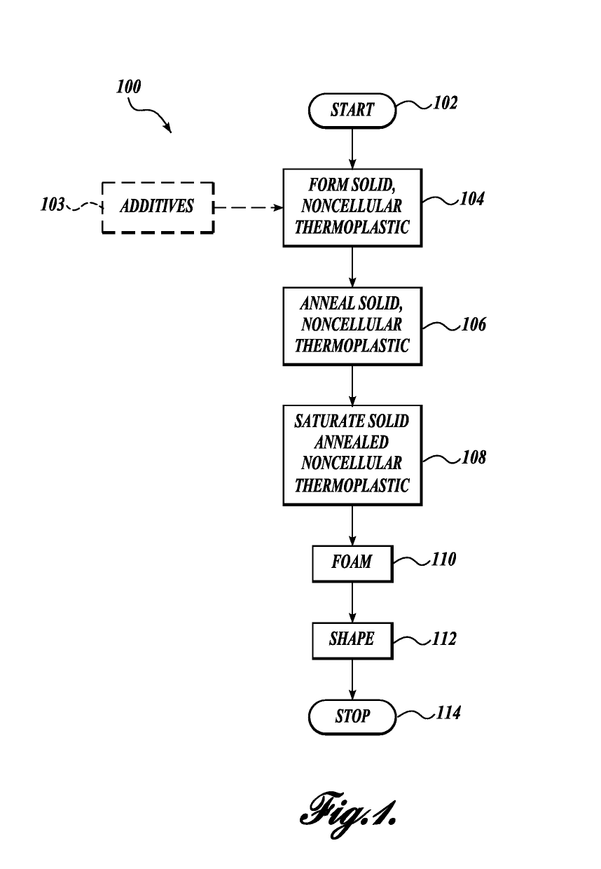

FIG. 1 discloses a method for creating solid-state foams in the above-identified cell size ranges.

The method 100 starts in block 102. From block 102, the method enters block 104. Block 104 is generally performed to provide the starting solid, noncellular material that is used in the disclosed solid-state foaming process. A starting material for the solid-state foaming process can be any shape, such as a film, sheet, formed product, or the like. In block 104, a solid, noncellular thermoplastic material is formed. This step can be performed by a number of processes. In block 104, the method of forming the solid, noncellular thermoplastic material may involve raising the temperature of the thermoplastic material above its melt point. For example, sheets and films of thermoplastic polyurethane can be created through an extrusion method. Generally, pellets or flakes of a thermoplastic material are fed to an extruder during which the pellets undergo melting. The melt is then passed through a die under pressure to create a sheet or shaped article, and following that, the sheet or shaped article is allowed to cool, or may be quenched to speed up the cooling process. Other methods may be used to form thermoplastic materials, such as a molding, casting, or cold-forming processes. The forming processes used in block 104 are generally well known methods. However, the methods may result in residual stresses in the formed materials due to rapid cooling and/or heating.

Block 104 is to be distinguished from block 110 discussed below. In block 110 a foam (cellular article) is formed by the application of heat (or rapid decrease in pressure) to cause thermodynamic instability, to a gas-saturated solid, noncellular thermoplastic material, wherein the temperature is kept below the melt temperature of the material. In block 104, heating may be performed to allow melting and shaping the material. Generally, no gas is introduced during the initial forming step, and therefore, the product of block 104 is a solid, noncellular thermoplastic material.

Once a solid, noncellular thermoplastic material has been formed in block 104, it can be allowed to cool and solidify and is then packaged into rolls, or sheets, or otherwise made into any other shape. The disclosed method uses the pre-formed solid products of block 104 as the starting materials for the solid-state foaming process disclosed herein.

From block 104, the method enters block 106. Block 106 is for annealing the solid, noncellular thermoplastic material. Annealing can be a heating step during which the temperature of the material is raised above the glass transition temperature of the thermoplastic material but below the melting temperature of the material. A glass transition temperature is a well-known term referring to the temperature or temperature range below which a thermoplastic material becomes somewhat like glass, being hard and possibly brittle. The glass transition temperature of virtually every thermoplastic material is published in the literature, or can be determined experimentally. In the annealing step, the time above the glass transition temperature can be on the order of hours to perhaps minutes. The annealing step duration and temperature may be dependent on the specific thermoplastic material being used, for example, whether the material is provided as a film or a thin sheet, a rolled thin sheet, or a solid block. The annealing step, block 106, may be done to reduce any stresses in the material induced during the initial manufacturing step, block 104. Annealing may include heating of the thermoplastic material, and maintaining, for a period of time, a temperature above the glass transition temperature. This is followed by slow cooling, which results in relieving some or all of the residual stress in the material.

From block 106, the method enters block 108. Block 108 is a step for saturating the annealed, solid, and noncellular thermoplastic material. “Saturate” as used herein means to allow the annealed, solid, noncellular thermoplastic material to take up or absorb a non-reacting gas, for example, nitrogen or carbon dioxide. The time and temperature of the saturation step, block 108, can depend on the particular thermoplastic material, and the saturation temperature and pressure. For example, a thin sheet of material may require less time than a solid block or a roll of a sheet of material. The gas-saturation step, block 108, may result in a fully gas-saturated (i.e., in gas equilibrium) material, or a partially gas-saturated material. The time and temperature for gas saturation to achieve a sufficient gas concentration may be determined via a series of trials, wherein the gas pressure and temperature are maintained. Samples are saturated in a pressure vessel and are weighed periodically to note the gas concentration. When the sample ceases to increase in weight, the sample is considered fully gas-saturated for the pressure and temperature conditions. Also, the temperature during the saturation process may be increased. In one particular embodiment described further below, saturation with attendant heating may be followed by the sudden release of pressure to create the foam.

In block 108, the thermoplastic material may be fully saturated or partially saturated. Generally, saturation takes place within a sealed vessel filled with the non-reacting gas at a pressure on the order of several atmospheres, such as 10 to 100 atmospheres, to speed the process of gas saturation into the material. When a thermoplastic material is removed from the saturation vessel, the material may then become supersaturated owing to the drop from several atmospheres to atmospheric pressure. After the saturation step, block 108, a period of gas desorption may follow. A purpose for allowing desorption is for the formation of a skin on the outer surfaces of the material. When taken out of a pressure vessel, the gas begins to escape the material from its outer surface, resulting in insufficient gas for foaming at the outer surfaces. Additionally, desorption may also be practiced in order to allow the gas concentration to reach a targeted gas concentration. Gas concentration is a parameter that may be adjusted to produce foams of varying cell characteristics.

From block 108, the method enters block 110. Block 110 is for heating the thermoplastic annealed, gas-saturated material to create a foam, i.e., a cellular material. The temperature in block 110 is raised at or above the glass transition temperature but is kept below the melt temperature of the material. Heating may be by way of immersing in a hot oil bath, or alternatively, by passing the gas-saturated material through an oven, or by heating simultaneously with a press. Reference may be made to prior publications on solid state foaming including, U.S. Pat. No. 5,684,055, U.S. Pat. No. 5,223,545, U.S. Pat. No. 7,923,104, and U.S. Pat. No. 8,092,626, all of which are expressly incorporated herein by reference.

In some embodiments, block 110, the heating step for creating a foam, may be omitted. In the case where heating block 110 is omitted, a foam may be created through the sudden release of pressure used in block 108. In some embodiments, depending on the characteristics of the material, the pressure saturation step block 108 can be practiced with or without attendant heating, and following a period of time to allow for saturation, the pressure is released suddenly to induce bubble nucleation and cell formation in the material. Further, as described above, the glass transition temperature of a thermoplastic material is reduced when saturated with a gas. In some materials, the glass transition temperature may be reduced sufficiently during the saturation process such that heating is no longer necessary, and bubble nucleation and cell formation is induced by the sudden release of pressure.

From block 110, the method enters block 112. Block 112 is optional. Block 112 is for shaping the foam material created in block 110, or in block 108 (if heating and block 110 is not required). Shaping may encompass various processes, such as cutting, stamping, molding, building, or assembling a useful product from the foam material. For example, the foams made in accordance with the disclosed method may be used as an insulation layer by themselves or in combination with other layers. In other embodiments, a continuous roll of film may be foamed as described above, and such film is then used in producing individual consumer articles, such as containers from the foamed sheet through a molding process.

“Thermoplastic” is a well-known term to designate polymers that can be repeatedly softened, melted, and then re-solidified. Thermoplastic materials have a softening point, i.e., the glass transition temperature, above which the polymer becomes flexible. Below the glass transition temperature, thermoplastics may have some degree of crystallinity. The thermoplastic materials that may be processed in accordance with the disclosed method are made from, for example, 100% by weight thermoplastic urethane, acrylonitrile butadiene styrene, polyamide, polybutadiene, polyethylene, polyethylene terephthalate, polymethyl methacrylate, polyester, polycarbonate, polylactic acid, polystyrene, or polyvinyl chloride. However, there are many other thermoplastics that may be processed in accordance with the methods herein. Polycarbonate, polystyrene, and polymethyl methacrylate may be preferred. Polycarbonate is of interest for several reasons. It is one of the most thoroughly studied amorphous polymers for solid-state microcellular processing. Furthermore, polycarbonate is an ideal material for creating a ‘clear’ nanofoam window due to its good mechanical strength. Polystyrene responds well to microcellular processing, and is one of the widely used foamed polymers for insulation applications. It is believed that employing an annealing process (heating and cooling steps), prior to saturation, a cell size range on the order of 10 nm or less can be accomplished by reducing the polymer surface energy via the annealing process.

The microcellular foams produced by the conventional solid-state foaming methods, without the annealing step as disclosed herein, typically have cell sizes in the 10-50 μm range, and are known as microcellular foams

In contrast, nanocellular foams, or nanofoams, have pore sizes in the range of several nanometers. To create nanofoams, a significant void-fraction is required. The introduction of an annealing step, prior to saturation, may provide a cell density many orders of magnitude higher than seen in microcellular foams, and may lead to the creation of nanofoams. The foams created via the disclosed process may produce foams with cell sizes less than 10 μm.

Nanofoams are hypothesized to offer mechanical properties that are superior to existing solid, noncellular materials and microcellular foams. Nanofoams may offer significant improvement in thermal insulation if the cells are smaller than the mean free path for molecular collisions, approximately 70 nm at room temperature. This improvement is based on the so-called Knudsen effect that occurs when the mean free path of the gas or air molecules inside the cell approaches the characteristic cell dimension. At this condition, the mechanism to transfer energy by molecular collisions will effectively not be operative, and the cells will behave as if there was vacuum inside them. Furthermore, when cells are sufficiently smaller than the wavelength of the visible light, the cells won't interfere with light. It may be possible to create clear but insulative window materials and packaging materials.

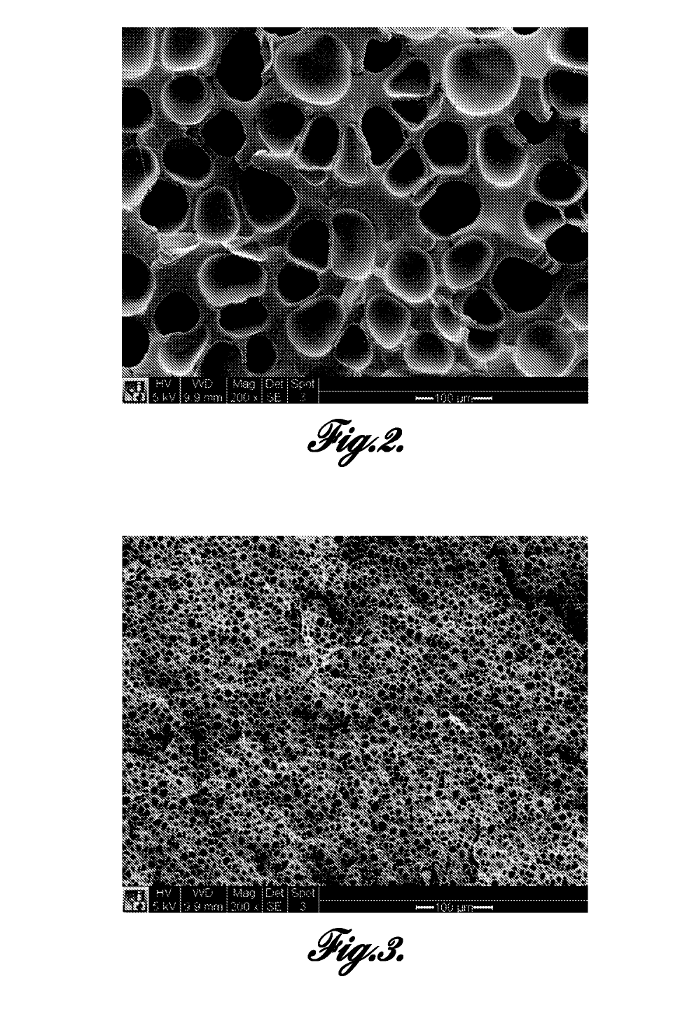

In example 1 below, a set of thermoplastic polyurethane (TPU) samples was not annealed, while a second set was annealed at 90° C. in an oven for two hours. Then, these two sets of samples were both saturated in a 3.65 MPa CO₂ environment at 80° F., and then foamed in a 115° C. silicone oil bath for 1 minute. Foamed samples were then examined using a scanning electron microscope (SEM). FIG. 2 shows the microstructure of a representative foam made from an unannealed sample, and FIG. 3 shows the microstructure of a representative foam made from an annealed sample. Note that FIG. 2 and FIG. 3 are taken at the same magnification. It was found that the annealing process increased the cell nucleation density by approximately 1,000 times, while the cell size was reduced by a factor of 10.

In order to understand the increased cell density, the classical nucleation theory which is the dominating theory for cell nucleation in microcellular foaming, was examined. The classical nucleation theory suggests that

$${N}_{0} = {C}_{0} {f}_{0} exp ( - \frac{Δ {G}_{crit}}{{k}_{B} T} )$$

where N₀ is the steady state nucleation rate, ΔG_{crit} is the free energy of critical nucleus formation (or activation energy), C₀ is the concentration of gas molecules, f₀ is the frequency factor, k_{B} is the Boltzmann's constant, and T is the absolute temperature in K. Since ΔG_{crit} appears in the exponent, it has a strong impact on cell nucleation. The ΔG_{crit} is further expressed as

$$Δ {G}_{crit} = \frac{16 π {σ}^{3}}{3 Δ {P}^{2}}$$

where σ denotes the surface energy of polymer-gas bubble, and ΔP in solid-state nucleation is taken to be the difference between gas saturation pressure and the atmospheric pressure. The exponent of the surface energy term is cubed, which indicates a strong relationship between surface energy and activation energy.

It appears that the annealing process reduces the polymer surface energy. From the above equations, the reduction in surface energy of the annealed samples may lead to a reduction in the activation energy for cell nucleation, resulting in a higher nucleation density.

In any event, the pre-saturation annealing process provides a highly effective means to increase the number of cells nucleated. This is expected to have far-reaching influence on microcellular processing, and on the continuing efforts to reduce the size of cells in polymer foams. In addition to much smaller cell sizes, the annealing step may have other advantages. Because of annealing prior to saturation, lower gas pressures needed for saturation are expected, making the process more cost-effective. However, while annealing (heating and cooling) is provided as one process to lower the surface energy, other process may be used.

Without ascribing to any particular theory, it is believed that annealing (by heating) is one of several possible means for reducing the surface energy of a thermoplastic material that may lead to increased cell nucleation density and smaller cell sizes. In other embodiments, the surface energy of the thermoplastic material may be reduced by the introduction of additives, such as fluorocarbon polymer particles or silicone particles, into the thermoplastic material. Such additives can be incorporated during the formation of the thermoplastic material. For example, additives, block 103, may be added during the thermoplastic forming step in block 104 of FIG. 1. Therefore, in the above described process of forming a cellular thermoplastic material, while in the solid phase, the annealing step (block 106) may be omitted. Instead, in block 104, the solid, noncellular thermoplastic material is formed with an additive, block 103, that lowers the surface energy of the material.

In addition to the solid state foaming process described in association with FIG. 1, the lowering of surface energy through an annealing step (or the introduction of additives) may also be practiced with an extrusion process. In an extrusion process, a thermoplastic material, usually in the form of pellets, is heated above the melting temperature within an extruder. While the material is in the melt state, a non-reacting gas is introduced into the melt while under pressure to saturate the melt. When the melt with the non-reacting gas exits the extruder through a die, the drop in pressure creates cells in the melt. The melt can be quenched thereafter to stop the foaming process. In one embodiment, the pellets may be annealed as described above, prior to introducing the pellets into the melt extruder. In another embodiment of an extrusion process, the pellets may be annealed, followed by saturating the pellets with the non-reacting gas, and then introduced into the extruder.

In some embodiments, a method includes heating a solid, noncellular, gas-unsaturated, thermoplastic material to a temperature greater than the material's glass transition temperature, and below the melting temperature, during which the thermoplastic material remains a solid. Then, allowing the material to cool. After the material has cooled, saturating the cooled thermoplastic material with a non-reacting gas to provide a gas-saturated material, during which the material remains a solid. Thereafter, heating the gas-saturated material below the melting temperature of the material so that the material remains a solid, and causes nucleation of bubbles, and creation of cells in the material.

In some embodiments, the material can be thermoplastic polyurethane.

In some embodiments, the material can be polycarbonate, polystyrene, or polymethyl methacrylate.

In some embodiments, the solid noncellular material is formed by melting prior to heating.

In some embodiments, the residual stresses as a result of melting and cooling are reduced by heating and slow cooling.

In some embodiments, the material can be a sheet or film.

Some embodiments of a method for creating a cellular thermoplastic material, include, forming a solid, noncellular thermoplastic material by melting and introducing an additive into the material, wherein the additive lowers a surface energy of the material; after the material has solidified, saturating the solid thermoplastic material with a non-reacting gas to provide a solid gas-saturated material; and heating the gas-saturated solid material below the melting temperature of the material so that the material remains a solid and causes nucleation of bubbles and creation of cells in the material.

Also disclosed are embodiments of a thermoplastic foam made by the methods above.

In some embodiments, the thermoplastic foam can have a relative density of about 54% to about 57%.

In some embodiments, the thermoplastic foam can have an average cell size less than 7 μm. In some embodiments, the thermoplastic foam can have an average cell size in the range of 5 μm to 10 μm.

In some embodiments, the thermoplastic foam can have a cell nucleation density greater than 3×10⁹ cells/cm³. In some embodiments, the thermoplastic foam can have a cell nucleation density that ranges from about 3×10⁹ cells/cm³ to about 6×10⁹ cells/cm³.

Some embodiments of a method for creating a foam from a solid thermoplastic material include applying a process to lower the surface energy of a solid, noncellular, gas-unsaturated, thermoplastic material, while the material remains a solid. After lowering the surface energy, saturating the solid thermoplastic material with a non-reacting gas during which the material remains a solid and provides a gas-saturated solid material. After saturating the solid thermoplastic material, inducing the nucleation of bubbles, and creation of cells in the gas-saturated solid material, while the material remains a solid.

In some embodiments, the method includes heating the gas-saturated material below the melting temperature of the material so that the material remains a solid, and causes the nucleation of bubbles, and creation of cells in the material.

In some embodiments, the solid noncellular gas-unsaturated material has been formed by a melting and cooling process that introduces residual stresses in the material, which are thereafter reduced.

In some embodiments, the process to lower the surface energy comprises heating the material above the glass transition temperature of the material, but lower than the melting temperature, and then cooling the material.

In some embodiments, the process to lower the surface energy is to introduce additives into the material, such as during the forming process.

### EXAMPLE 1

### Introduction

Variability in cell size and cell nucleation density was investigated along a 100 foot TPU roll under controlled laboratory conditions. The material used in all experiments of Example was 42D hardness TPU.

Experimental Method

Saturation Procedure

All samples of TPU were cut into one inch circles using a metal punch and individually labeled. For CO₂ saturation, samples were contained in a metal pressure vessel with controlled temperature and pressure. High pressure CO₂ was provided by a Praxair gas cylinder to a lab-grade purity. The vessel pressure was regulated by an Omega process controller between 3.65 and 3.67 MPa. The temperature was regulated by an external electrical heating pad on the surface of the pressure vessel and an internal temperature probe. The heating pad was controlled by a tuned Omega temperature controller set to 80° F. For all experiments, the samples were wrapped in paper to allow even exposure to CO₂ and placed in the pre-heated pressure vessel. The samples were not dried prior to this. The pressure vessel was pressurized and purged of any residual air. These conditions were maintained for at least 8 hours to ensure full saturation of the samples.

Annealing Procedure

Select samples were annealed prior to saturation for the purposes of Experiment II. These samples were placed in a convection oven at 90° C. for 2 hours and then cooled down to room temperature. The annealed samples were then allowed to rest at room temperature for 2 days before any further steps were taken.

Foaming Procedure

After the saturation step was complete, the pressure was released and the samples were foamed in a Thermo-Haake B5 circulating silicone oil bath set to 115° C. for 60 seconds. In all experiments, the time between the release of pressure and the introduction to the heat bath was set to 120 seconds. After removal from the heat bath, the samples were quenched in room temperature water to stop the foaming process, washed in detergent and rinsed to remove any residual silicone oil. The samples were allowed to sit for at least 2 days before any analysis was conducted.

Relative Density Measurement

Relative density is a ratio of the foam density to the virgin material density. The relative density of each sample was measured by displacement in accordance with ASTM D792. A Mettler AE240 scale was used in conjunction with a density measurement apparatus to perform these experiments using distilled water as the displacing liquid. Two dry mass measurements and three wet mass measurements were taken for each sample to ensure accuracy. Relative density is equivalent to 1 minus the void fraction.

Microstructure Characterization

Scanning electron microscopy (SEM) was employed to characterize the microstructure of the foamed samples. The FEI Sirion SEM at the Nanotech User Facility (NTUF) at the University of Washington was used in this experiment. Samples are first freeze fractured using liquid nitrogen to produce a fracture surface that accurately reflects the microstructure. The resulting samples are then mounted in stages and sputter coated with Au/Pd for 90 seconds using a SPI sputter module controller. Finally, the sputtered samples were investigated in the SEM with an accelerating voltage of 5 kV, a spot size of 3, and a working distance around 7.5 mm.

Cell density, N_{f}, is defined as the number of cells per cm³ of the foam. It is calculated by

$${N}_{f} = {[ \frac{{nM}^{ 2}}{A} ]}^{3 / 2}$$

where n is the number of cells in the micrograph, A is the area of the selected region on the SEM image, and M is the magnification.

Cell nucleation, N₀, density is defined as the number of cells per cm³ of the original, unfoamed polymer. It is calculated by

$${N}_{0} = \frac{{N}_{f}}{1 - {ρ}_{rel}}$$

where ρᵣₑₗ is the relative density of the foam.

To calculate the cell density, the total number of cells in a measured area is counted. Generally, a SEM image with greater than or equal to 100 cells is sufficient for obtaining an accurate cell density.

Experiments

Experiment I

The goal of Experiment I was to establish a reference level of variability in one roll of raw TPU. 1-inch diameter unannealed samples were taken from the left, right, and center of each odd-numbered sheet in a 32-sheet roll. Thus, 48 total samples were randomly distributed into six 8-sample batches, foamed, and characterized.

Experiment II

The goal of Experiment II was to study the effect of pre-foaming annealing on TPU solubility and foaming. To investigate the solubility of CO₂ in annealed TPU, annealed samples were saturated and the final concentration was measured and compared to control samples of unannealed TPU. 1 minute of desorption time was allowed between removal of the samples from the pressure vessel and measurement of the final concentration.

To investigate the effect of annealing on the final foam structure, annealed and unannealed samples were foamed and the resulting microstructures compared. Two 1-inch diameter samples were taken from the center of 10 even numbered rolls. These samples were labeled and separated into two groups of unannealed and annealed samples.

Experiment III

The goal of Experiment III was to investigate the variation of average cell size and cell nucleation density within one sample to provide insight into the results of the previous experiments. Two foamed samples were used for this investigation, one annealed and one unannealed. SEM images were taken from 10 different locations along the centerline of one fracture surface from a foamed sample over a distance of 7 mm. Similarly, SEM images were taken from a second foamed sample over a distance of 8 mm. The resulting images were characterized and analyzed to show the local variation of microstructure in each sample.

Results

Experiment I

The results from Experiment I are summarized in Table 1. This table compares the average results from 48 total samples from the left, center, and right sides of the roll.

TABLE 1
Experiment I Results Summary

| | | Left | Center | Right | All |
|---|---|---|---|---|---|
| Average Cell | Average | 62.7 | 64.7 | 69.7 | 65.7 |
| Size (μm) | St. Dev. | 9.2 | 7.2 | 10.4 | 9.9 |
| Relative | Average | 52.04% | 53.03% | 52.47% | 52.51% |
| Density | St. Dev. | 1.33% | 1.74% | 2.12% | 1.77% |
| Nucleation | Average | 5.25E+06 | 4.36E+06 | 3.84 + 06 | 4.48 + 06 |
| Density | St. Dev. | 2.31 + 06 | 1.88 + 06 | 1.68 + 06 | 2.02 + 06 |
| (cells/cm³) | | | | | |

Experiment II

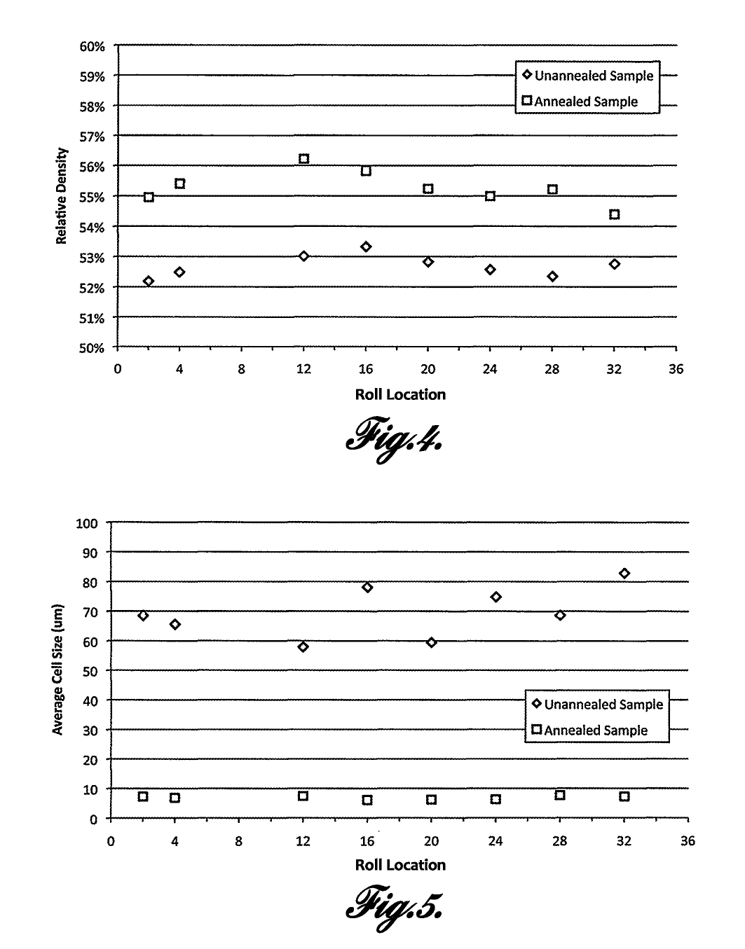

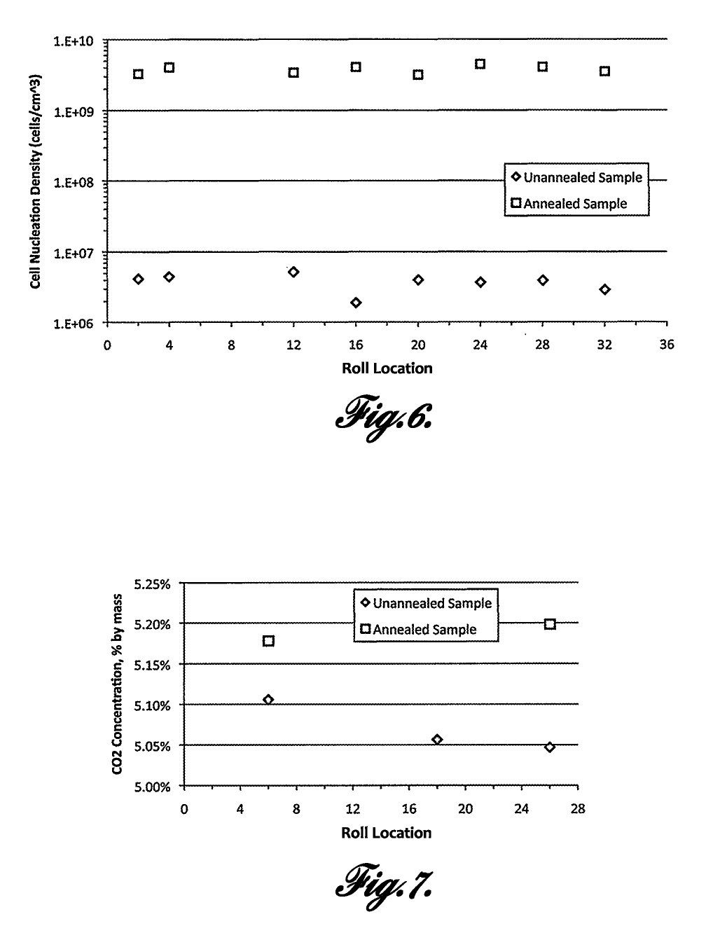

A summary of the results of the foaming portion of Experiment II can be found in Table 2. FIG. 4 shows the relative density of the Experiment II samples. The X-axis in FIGS. 4 through 7 indicates the distance along the length of a roll of material from which the samples were taken. For example, when a roll is 100 feet in length, each number on the X-axis can represent units of 3 feet. The average relative density of all 8 annealed samples (55.3%) is about 2.6% higher than that of unannealed samples (52.7%). FIG. 5 shows the average cell size of the Experiment II samples. The average cell size of the annealed samples was 62.6 μm smaller than the average cell size of the control samples. FIG. 6 compares the nucleation density in annealed and unannealed samples. The nucleation density of the annealed TPU is about three orders of magnitude greater than the control, on average.

TABLE 2
Experiment II foaming results summary

| | Relative | | Average Cell | | Nucleation | |
|---|---|---|---|---|---|---|
| | Density | | Size (μm) | | Density | |
| | Aver- | St. | Aver- | St. | Aver- | St. |
| | age | Dev. | age | Dev. | age | Dev. |
| Un- | 52.69% | 0.37% | 69.5 | 8.7 | 3.795E+06 | 9.984E+05 |
| annealed | | | | | | |
| Annealed | 55.28% | 0.55% | 6.9 | 0.6 | 3.757E+09 | 4.667E+08 |

Experiment III

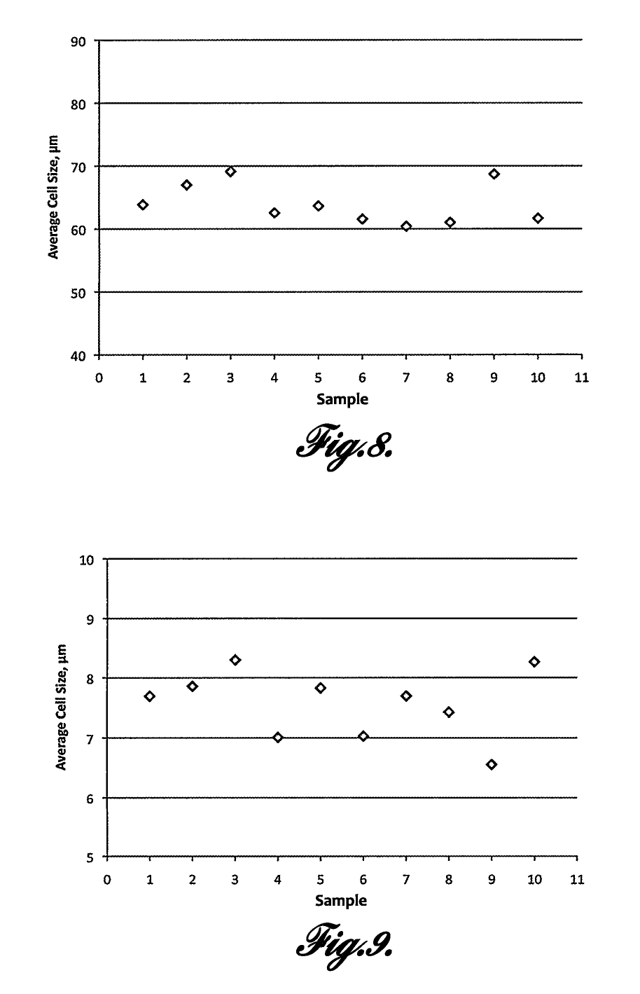

FIG. 8 and FIG. 9 show the local variation in average cell size for an unannealed and annealed sample, respectively. The average cell size for the unannealed sample was 64.0 μm with a standard deviation of 3.2 μm. The average cell size for the annealed sample was 7.6 μm with a standard deviation of 0.6 μm.

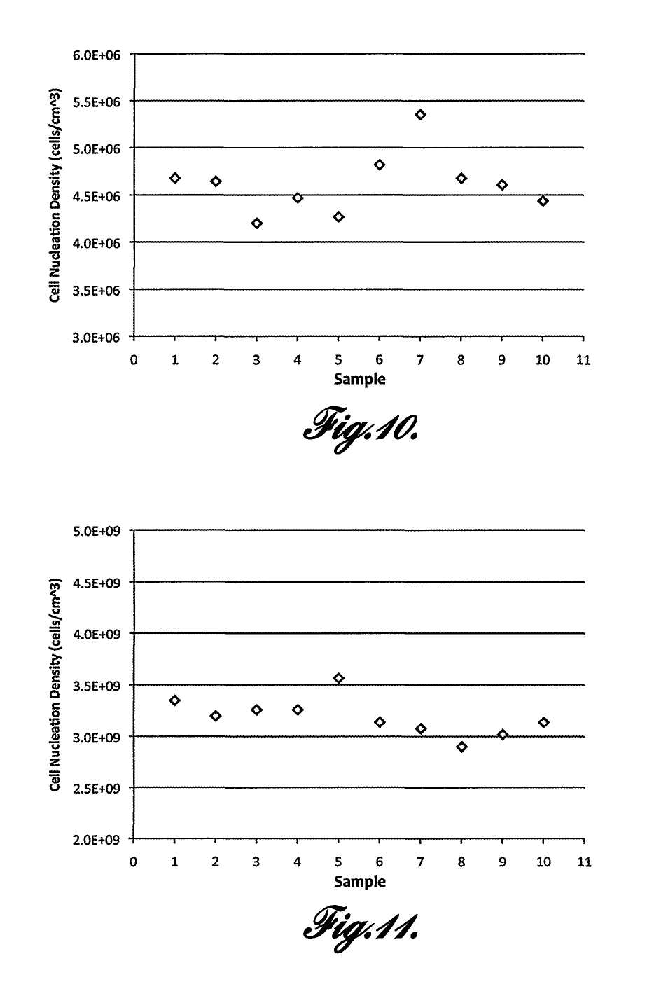

FIG. 10 and FIG. 11 show the local variation in cell nucleation density for the unannealed and annealed sample, respectively. The average measured cell nucleation density for the unannealed sample was 4.62×10⁶ cells/cm³ with a standard deviation of 3.23×10⁶ cells/cm³. The average measured cell nucleation density for the annealed sample was 3.19×10⁹ cells/cm³ with a standard deviation of 1.84×10⁸ cells/cm³.

Discussion

The results of these experiments show that even in a tightly controlled laboratory foaming process, the microstructure of the resulting material has some measurable variability, both locally and across the extruded roll. In addition, along the width of the roll, a trend of lower nucleation density and higher cell size has been identified from left to right. No significant microstructure trends were observed in the roll length direction.

These results clearly show that pre-saturation annealing of this material has a very significant effect on the microstructure of the resulting foam. Annealed samples absorb slightly more gas at saturation than unannealed samples. The annealing process also produced foams with three orders of magnitude larger nucleation density and one order of magnitude smaller average cell size. In addition, the global and local variability of average cell size and nucleation density was significantly reduced in the annealed samples. It is possible that this is due to the intrinsic nature of smaller cell size and larger nucleation density foams, however.

Experiment III quantifies the local variability in cell size and nucleation density in one foamed sample as measured using this characterization process. This provides insight into the results of Experiments I and II, as this local variability is present in each of the data points and contributes to the global variability throughout the roll.

### EXAMPLE 2

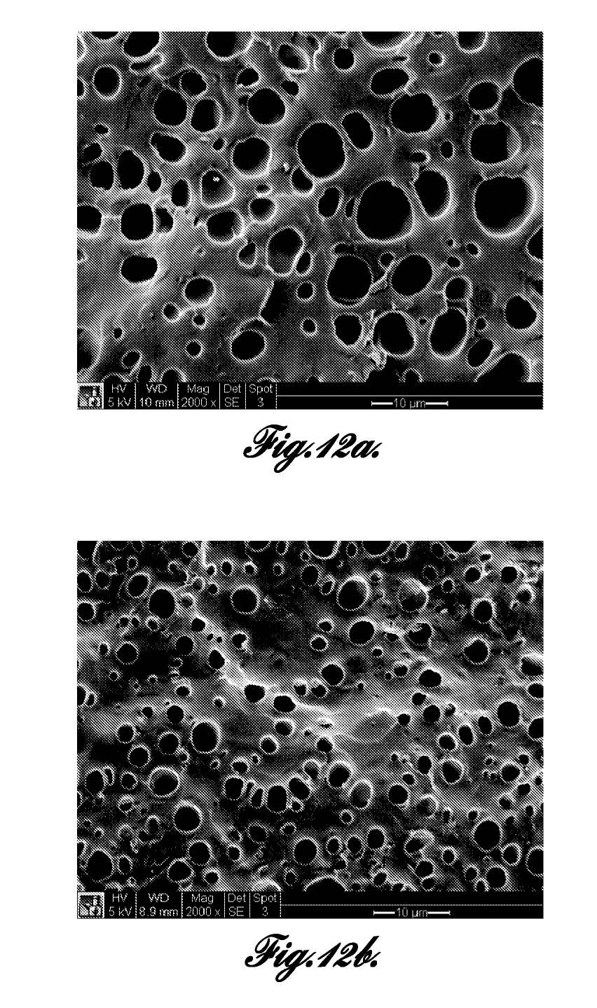

Experiments were performed using the following polymers: 42D (Shore hardness) TPU (thermoplastic polyurethane), 72D (Shore hardness) TPU, PC (polycarbonate), and PS (polystyrene). For each polymer, a set of unannealed samples and another set of annealed samples were foamed under the same conditions. Annealing temperature for a specific polymer was selected based on its glass transition temperature. The resulting foam microstructures are shown in FIGS. 12A, 12B, 13A, 13B, 14A, 14B, 15A, and 15B.

### Results

42D TPU

FIGS. 12A and 12B show the comparison between the microstructure of foamed samples starting from unannealed and annealed 42D TPU materials, respectively. Samples were annealed at 90° C. for 2 hours. Processing conditions for foaming included a saturation pressure of 5 MPa at room temperature, a foaming temperature of 80° C., and a foaming time of 1 minute. Under these processing conditions, the annealed sample (FIG. 12B) resulted in about 3 times the cell nucleation density as compared to that of unannealed 42D TPU (FIG. 12A).

72D TPU

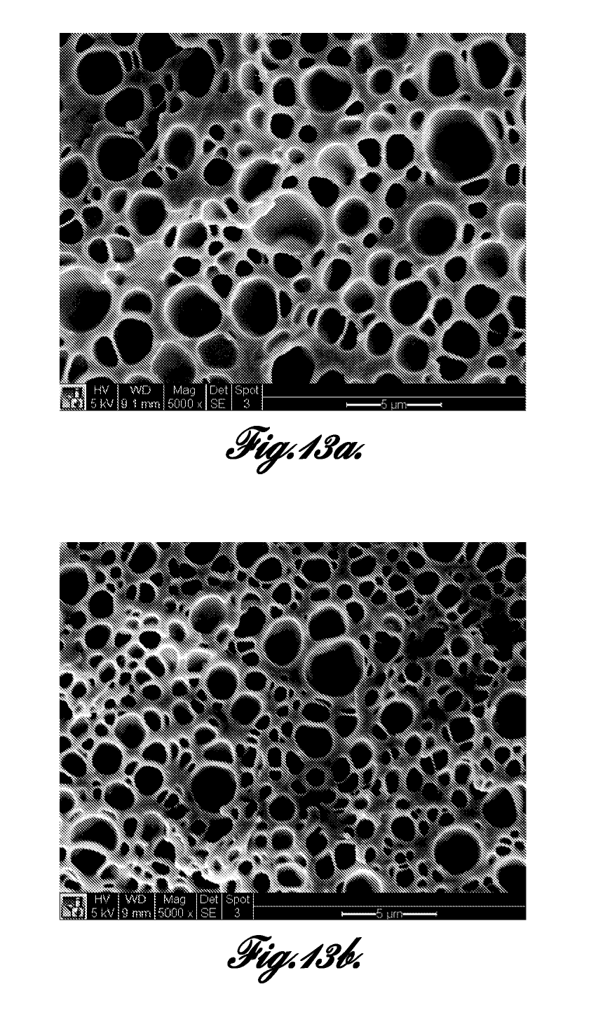

FIGS. 13A and 13B show the comparison between the microstructure of foamed samples starting from unannealed and annealed 72D TPU materials, respectively. Samples were annealed at 90° C. for 2 hours. Processing conditions for foaming included a saturation pressure of 3 MPa at room temperature, a foaming temperature of 150° C., and a foaming time of 1 minute. The annealed sample (FIG. 13B) resulted in about 3 times the cell nucleation density as compared to that of unannealed 72D TPU (FIG. 13A).

PC

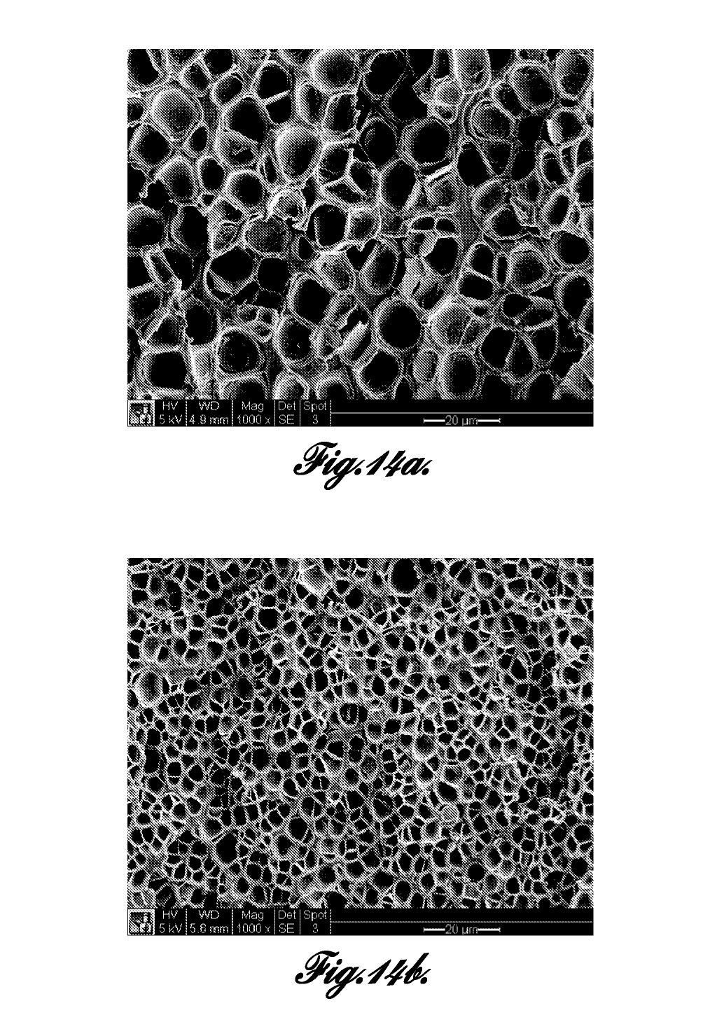

The effect of thermal annealing on PC depended on the annealing temperature used. At lower annealing temperatures of 140° C., 150° C. and 180° C., unannealed PC and annealed PC showed generally the same microstructure. At the higher annealing temperature of 250° C., however, the annealed sample (FIG. 14B) resulted in about 10 times an increase in the cell density and 3 times a decrease in cell size as compared to the unannealed sample (FIG. 14A). The samples were annealed at 250° C. for 2 hours. Processing conditions for foaming included a saturation pressure of 3 MPa at room temperature, a foaming temperature of 120° C., and a foaming time of 2 minutes.

PS

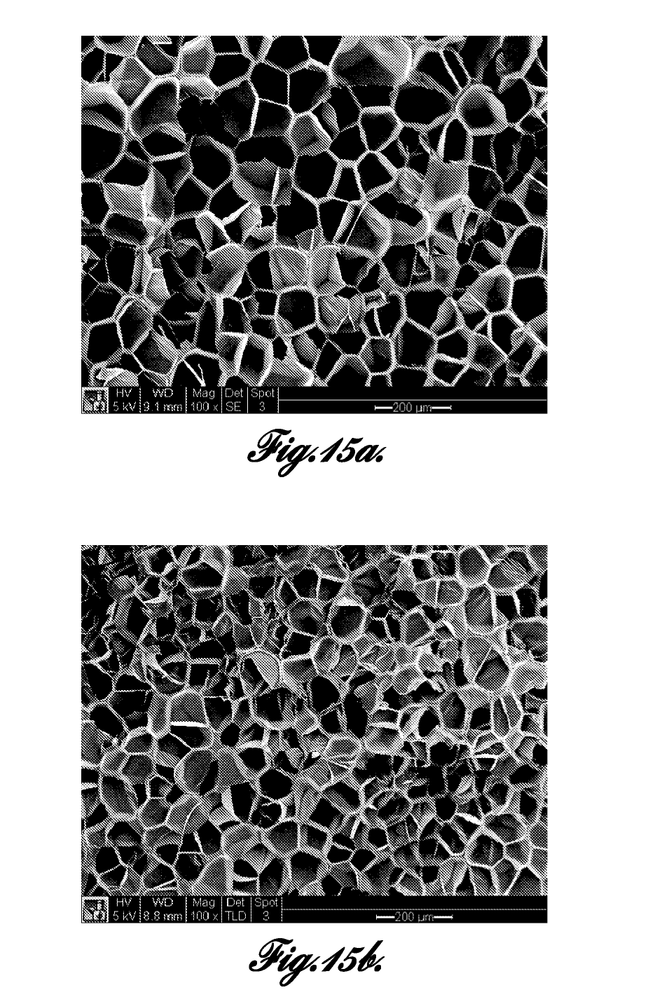

FIGS. 15A and 15B show the comparison between the microstructure of foamed samples starting from unannealed and annealed polystyrene materials, respectively. Samples were annealed at 77° C. for 1.5 hours. Processing conditions for foaming included a saturation pressure of 1 MPa at room temperature, a foaming temperature of 80° C., and a foaming time of 2 minutes. The annealed sample (FIG. 15B) resulted in about 1.5 times the cell nucleation density compared to that of unannealed PS (FIG. 15A).

For the polymer systems investigated in Examples 1 and 2, thermal annealing can be used to increase the cell nucleation densities. The extent of cell nucleation density increase depends on the polymer systems and the specific processing conditions. The largest increase in cell nucleation density is on the order of 1000 times in 42D TPU (See Example 1).

While the preferred embodiment of the invention has been illustrated and described, it will be appreciated that various changes can be made therein without departing from the spirit and scope of the invention.

1. A peptide selected from the group consisting of:
a) a peptide comprising an amino acid sequence at least 78% identical to the amino acid sequence of SEQ ID NO: 2,
b) a peptide comprising an amino acid sequence at least 82% identical to the amino acid sequence of SEQ ID NO: 2,
c) a peptide comprising an amino acid sequence at least 86% identical to the amino acid sequence of SEQ ID NO: 2,
d) a peptide comprising an amino acid sequence at least 91% identical to the amino acid sequence of SEQ ID NO: 2,
e) a peptide comprising an amino acid sequence at least 95% identical to the amino acid sequence of SEQ ID NO: 2, and
f) a peptide comprising an amino acid sequence that is 100% identical to the amino acid sequence of SEQ ID NO: 2,
wherein the peptide does not comprise the amino acid sequence of SEQ ID NO: 1.

2. The peptide of claim 1, wherein the peptide comprises an amino acid sequence selected from the group consisting of the amino acid sequence of SEQ ID NO: 3, the amino acid sequence of SEQ ID NO: 4, the amino acid sequence of SEQ ID NO: 5, the amino acid sequence of SEQ ID NO: 6, the amino acid sequence of SEQ ID NO: 7, the amino acid sequence of SEQ ID NO: 8, the amino acid sequence of SEQ ID NO: 9, the amino acid sequence of SEQ ID NO: 10, the amino acid sequence of SEQ ID NO: 11, the amino acid sequence of SEQ ID NO: 12, the amino acid sequence of SEQ ID NO: 13, the amino acid sequence of SEQ ID NO: 14, the amino acid sequence of SEQ ID NO: 15, the amino acid sequence of SEQ ID NO: 16, the amino acid sequence of SEQ ID NO: 17, the amino acid sequence of SEQ ID NO: 18, the amino acid sequence of SEQ ID NO: 19, the amino acid sequence of SEQ ID NO: 20, the amino acid sequence of SEQ ID NO: 21, the amino acid sequence of SEQ ID NO: 22, and the amino acid sequence of SEQ ID NO: 23.

3. An antibody or antibody fragment comprising the peptide of claim 1, wherein the peptide is a heavy chain complementarity determining region (CDR).

4. The antibody or antibody fragment of claim 3, further comprising at least one additional heavy chain CDR.

5. The antibody or antibody fragment of claim 4, wherein the at least one additional heavy chain CDR comprises the amino acid sequence of SEQ ID NO: 25.

6. The antibody or antibody fragment of claim 5, further comprising a second additional heavy chain CDRs.

7. The antibody or antibody fragment of claim 6, wherein the second additional heavy chain CDRs comprises the amino acid sequence of SEQ ID NO: 26.

8. The antibody or antibody fragment of any of claim 3, further comprising at least one light chain CDR.

9. The antibody or antibody fragment of claim 8, wherein the at least one light chain CDR comprises the amino acid sequence of SEQ ID NO: 27.

10. The antibody or antibody fragment of claim 9, further comprising a second light chain CDR.

11. The antibody or antibody fragment of claim 10, wherein the second light chain CDR comprises the amino acid sequence of SEQ ID NO: 28.

12. The antibody or antibody fragment of claim 11, further comprising a third light chain CDR.

13. The antibody or antibody fragment of claim 12, wherein the third light chain CDR comprises the amino acid sequence of SEQ ID NO: 29.

14. A method of treating a Hendra virus or Nipah virus infection comprising administering the antibody or antibody fragment of claim 3 to a subject which has been infected with Hendra or Nipah virus.

15. A method of reducing the likelihood of a subject developing a disease caused by Hendra virus or Nipah virus, the method comprising administering the antibody or antibody fragment of claim 3 to a subject prior to Hendra virus infection or Nipah virus infection.

16. A nucleic acid encoding the peptide of claim 1.

17. A vector comprising the nucleic acid of claim 16.

18. A host cell comprising the vector of claim 17.

19. A method of making a peptide comprising an amino acid of SEQ ID NO: 2, SEQ ID NO: 3; SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23, the method comprising culturing the host cell of claim 18 under conditions suitable for protein expression and isolating the peptide.

20. An antibody that binds to the four hydrophobic pockets of the G glycoprotein head of Hendra virus or Nipah virus.
